(19) United States
(12) Patent Application Publication
(10) Pub. No.: US 2026/0235620 A1
(43) Pub. Date: Aug. 13, 2026

(54) Glycan Analysis of Proteins in Cells

(71) Applicants: MUSC Foundation for Research Development, Charleston, SC (US); Van Andel Research Institute, Grand Rapids, MI (US)

(72) Inventors: Anand Mehta, Mt. Pleasant, SC (US); Richard R. Drake, Charleston, SC (US); Brian Haab, Jenison, MI (US); Peggi M. Angel, Charleston, SC (US)

(21) Appl. No.: 19/381,523

(22) Filed: Nov. 6, 2025

Related U.S. Application Data
(63) Continuation of application No. 17/059,660, filed on Jun. 3, 2019, now abandoned.
(60) Provisional application No. 62/679,202, filed on Jun. 1, 2018.

(51) Int. Cl. G01N 33/68

(52) U.S. Cl. CPC G01N 33/6848; G01N 2440/38; G01N 2570/00

(57) ABSTRACT

The present invention provides methods and compositions for glycan analysis of complex solutions, including proteins and cells in a biological sample. The method includes the preparation of substrates for the capture of proteins and cells for multiplexed analysis. Cells and proteins may be captured by antibody arrays, culture, or direct deposition. The invention further relates to the use of protein and cell glycan analysis in the diagnosis and screening of disease states and disease progression.

### CROSS-REFERENCE TO RELATED APPLICATIONS

This application claims priority to U.S. Provisional Patent Application No. 62/679,202, filed Jun. 1, 2018, the contents of which are incorporated by reference herein in its entirety.

### STATEMENT REGARDING FEDERALLY SPONSORED RESEARCH OR DEVELOPMENT

This invention was made with government support under Grant No. R21CA225474 provided by the National Cancer Institute. The government has certain rights in the invention.

### BACKGROUND OF THE INVENTION

Changes in the N-linked glycosylation of cells surface and secreted proteins are known to occur in many cancers. Indeed, these glycans often mediate the interactions between the cancer cell and its environment. Almost every currently used biomarker for cancer is either a glycoprotein, such as carcinoembryonic antigen (CEA) or a glycan itself, such as CA-19-9. However, due to the inherent difficulty in glycoproteomics, where the identity of a protein and the glycans on that protein are deduced, most glycoprotein biomarker assays target either just the protein itself, such as is the case with CEA, or just the glycan itself, which is the case with CA-19-9. Recent work has shown that specific glycans on specific proteins can act as biomarkers of cancer and often they are better markers than the protein alone. However, obtaining this glycan information is laborious and difficult.

Currently, glycans are examined on either individual proteins or in large protein pools, such as serum or urine, where glycan information is obtained but protein information is lost. Thus, the tradeoff is that glycan information can be obtained for a few proteins analyzed one by one with site of glycan attachment, or data can be obtained for groups of proteins or glycans (but not both together). One approach that attempts to address this issue is the use of antibody lectin arrays. In this case, antibodies to specific proteins are spotted onto glass slides and the glycans on the captured glycoproteins are interrogated with sugar binding proteins (lectins). While this data does provide evidence for specific structural motifs, it offers no true insight into the glycan diversity on a protein nor does it offer true structural information.

There remains a need for a method to deliver structural glycan information for specific glycoproteins in complex solutions. The present invention satisfies this need.

### SUMMARY OF THE INVENTION

In one aspect, the present invention provides a method for glycan analysis of at least one sample, the method comprising the steps of: providing a substrate having a surface spotted with a plurality of antibodies; incubating the substrate in a blocking solution; incubating the substrate in at least one sample; spraying the substrate with an enzymatic releasing solution; and scanning the substrate by mass spectrometry to detect and identify the presence of glycans.

In one embodiment, the at least one sample comprises at least one protein solution. In one embodiment, the at least one sample comprises at least one population of cells. In one embodiment, the at least one population of cells is incubated in a fixing and rinsing agent prior to the step of spraying the substrate with an enzymatic releasing solution. In one embodiment, the fixing and rinsing agent is selected from the group consisting of: formalin, Carnoy's solution, paraformaldehyde, an ethanol-based fixative, and a polyethylene glycol-based fixative.

In one embodiment, the substrate is a glass or plastic microscope slide or multiwell plate. In one embodiment, the blocking solution is a serum. In one embodiment, the serum is 1% BSA in PBS and detergent. In one embodiment, the blocking solution is removed with a wash step comprising 3×PBS baths and 1×water bath. In one embodiment, the at least one sample is incubated in a humidity chamber at room temperature for two hours. In one embodiment, the enzymatic releasing solution comprises PNGase F.

In one embodiment, the mass spectrometry is selected from the group consisting of: matrix-assisted laser desorption/ionization imaging Fourier transform ion cyclotron resonance (MALDI-FTICR) mass spectrometry, matrix-assisted laser desorption/ionization time of flight (MALDI-TOF) mass spectrometry, scanning microprobe MALDI (SMALDI) mass spectrometry, infrared matrix assisted laser desorption electrospray ionization (MALD-ESI) mass spectrometry, surface-assisted laser desorption/ionization (SALDI) mass spectrometry, desorption electrospray ionization (DESI) mass spectrometry, secondary ion mass spectrometry (SIMS) mass spectrometry, and easy ambient sonic spray ionization (EASI) mass spectrometry. In one embodiment, the scanning step is preceded by a step of spraying the substrate with a MALDI matrix material. In one embodiment, the MALDI matrix solution is selected from the group consisting of: 2,5-dihydroxybenzoic acid, α-cyano-4-hydroxycinnamic acid, sinapinic acid, 1,5-diaminonaphthalene, and 9-aminoacridine.

In one embodiment, the plurality of antibodies specifically bind to a protein selected from the group consisting of: A1AT, fetuin-A, hemopexin, Apo-J, LMW Kininogen, HMW Kininogen, apo-H, transferrin, IgG, IgM, IgA, fibronectin, laminin, ceruloplasmin, fibulin, angiotensinogen, Fibrillin-1, TIMP1, thrombospondin 1, galectin-3 binding protein, complement C1 R, clusterin, galectin 1, alpha-2-macroglobulin, Vitamin D binding protein, histidine rich glycoprotein, histidine rich glycoprotein, CD109, CEA, Cathepsin, AFP, GP731, and combinations thereof. In one embodiment, the antibodies are useful in detecting the presence of hepatocellular carcinoma.

In another aspect, the present invention provides a method for glycan analysis of at least one population of cells, the method comprising the steps of: adhering at least one population of cells to a surface of a substrate; fixing and rinsing the at least one population of cells; spraying the substrate with an enzymatic releasing solution; and scanning the substrate by mass spectrometry to detect and identify the presence of glycans.

In one embodiment, the at least one population of cells is adhered by culturing, deposition, swabbing, smearing, or centrifugation. In one embodiment, the fixing and rinsing agent is selected from the group consisting of: formalin, Carnoy's solution, paraformaldehyde, an ethanol-based fixative, and a polyethylene glycol-based fixative.

In one embodiment, the substrate is a glass or plastic microscope slide or multiwell plate. In one embodiment, the substrate surface includes one or more of: an indium tin oxide coating, a gelatin coating, a collagen coating, a poly-l-lysine coating, a poly-ornithine coating, an extracellular matrix coating, a protein coating, and surface ionization. In one embodiment, the enzymatic releasing solution comprises PNGase F.

In one embodiment, the mass spectrometry is selected from the group consisting of: matrix-assisted laser desorption/ionization imaging Fourier transform ion cyclotron resonance (MALDI-FTICR) mass spectrometry, matrix-assisted laser desorption/ionization time of flight (MALDI-TOF) mass spectrometry, scanning microprobe MALDI (SMALDI) mass spectrometry, infrared matrix assisted laser desorption electrospray ionization (MALD-ESI) mass spectrometry, surface-assisted laser desorption/ionization (SALDI) mass spectrometry, desorption electrospray ionization (DESI) mass spectrometry, secondary ion mass spectrometry (SIMS) mass spectrometry, and easy ambient sonic spray ionization (EASI) mass spectrometry. In one embodiment, the scanning step is preceded by a step of spraying the substrate with a MALDI matrix material. In one embodiment, the MALDI matrix solution is selected from the group consisting of: 2,5-dihydroxybenzoic acid, α-cyano-4-hydroxycinnamic acid, sinapinic acid, 1,5-diaminonaphthalene, and 9-aminoacridine.

In another aspect, the present invention relates to a kit for glycan analysis of protein samples, comprising: at least one substrate, each substrate having a surface spotted with a plurality of antibodies; at least one blocking solution; at least one enzymatic releasing solution; and at least one MALDI matrix material.

In one embodiment, the substrate is a glass or plastic microscope slide or multiwell plate. In one embodiment, the blocking solution is a serum. In one embodiment, the serum is 1% BSA in PBS and detergent. In one embodiment, the enzymatic releasing solution comprises PNGase F. In one embodiment, the MALDI matrix solution is α-cyano-4-hydroxycinnamic acid.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1 depicts an overview of an exemplary method of the present invention. Antibodies are coated onto glass slides as in a traditional antibody microarray. In a first step, the entire slide is sprayed with recombinant PNGase F to remove the inherent glycosylation of the antibodies and following a 1-hour incubation period, the slide is washed in 1×PBS. Subsequently, samples are added to the whole slide (such as serum mixtures or protein), washed again and sprayed with recombinant PNGase F. Matrix is added and MALDI-FTICR MS performed. Structural glycan information is obtained for each captured protein, spot by spot.

FIG. 2 is a flowchart of an exemplary method of the present invention.

FIG. 3 is a flowchart of another exemplary method of the present invention.

FIG. 4 depicts an example of glycan-MALDI-imaging. A primary liver cancer of the genetic sub-type S3 (well differentiated; slow growing) were analyzed by MALDI-mass imaging. A number of glycans can be observed that are spatially located on the slide (tissue).

FIG. 5A through FIG. 5C depict the detection of anti-body captured protein. (FIG. 5A) N-linked glycan profile of A1AT by normal phase HPLC. The major glycans are indicated. (FIG. 5B) A1AT spotted onto glass slides and N-linked glycan detected by MALDI-FTICR MS. The core fucosylated bi-antennary glycan (m/z=1809.639) is shown. (FIG. 5C) The core fucosylated bi-antennary glycan detected following capture of A1AT by an antibody. As a control, antibody to human fetuin-A was also used. Values (0, 1.0, 0.1, 0.01, 0.001, 0.0001) are in μg of protein added.

FIG. 6 depicts the results of experiments detecting N-glycans from captured IgG. N-glycans are listed in order of peak intensities (e.g., abundance) (left). HPLC profiles from IgG are shown in comparison (right).

FIG. 7 depicts an example of a multiplexed array slide. Each quadrant consists of 32 antibodies to individual proteins. The quadrants can be identical and can be treated differently.

FIG. 8 depicts deglycosylation of array spots. Preliminary evidence for the de-glycosylation of antibody. The left image shows an array without PNGase Fand the right image shows an array with PNGase F. The slides have printed antibody to MUC5AC, MUC3, endorepellin, and biotinylated IgG. Fucose on the attached antibody was detected with the Ralstonia solanacearum lectin. After treatment with PNGase F, all inherent lectin binding is abolished. Signal from biotinylated IgG acts as a control for loading.

FIG. 9 depicts another multiplexed array slide and a schematic of the method of the present invention.

FIG. 10 depicts the results of capture of de-sialyated, denatured A1AT spotted directly to antibody.

FIG. 11 depicts the capture of IgG from 7 μL samples, one antibody spotted per well.

FIG. 12A through FIG. 12D depict the results of experiments demonstrating N-glycan profiling of endothelial cell (EC) single cell layers through simplified MALDI MS workflows. (FIG. 12A) Before delipidation. (FIG. 12B) After delipidation. (FIG. 12C) Complex N-glycan profiles obtained from a single cell layer of EC. (FIG. 12D) Image data of cell chambers. Note that G1 peak is also seen (at a lower level) in cell media consistent with known IgG patterns.

FIG. 13A through FIG. 13D depict the results of experiments demonstrating stable isotopic labeling in cell culture (SILAC) detected by IMS. (FIG. 13A) 15N label was applied to 10,000 Aortic endothelial cells cultured for 1 week in either 14N or 15N glutamine media. (FIG. 13B) 15N is incorporated in all 4 GlcNAc residues of a complex N-glycan, a mass shift of 3.9895 Da. (FIG. 13C) IMS detection of labeled N-glycan G1F. (FIG. 13D) Single spectra demonstrating strong detection of labeled N-glycan.

FIG. 14A through FIG. 14C depict a diagram illustrating a novel workflow for MALDI imaging of N-glycans released from immunocaptured glycoproteins. (FIG. 14A) Antibody array is created by spotting of antibodies at 200 ng per 1.5 μL spot to a nitrocellulose-coated slide. The slide is blocked with BSA and then sample is added for capture of glycoproteins by their respective antibodies. (FIG. 14B) Slide array is prepared for MALDI MSI by enzymatic release of N-glycans in a localized manner followed by matrix application. (FIG. 14C) A MALDI FT-ICR MS is used for imaging the slide to obtain an overall spectra and individual images for each m/z peak, showing the abundance of each N-glycan in two-dimensions across the array.

FIG. 15 depicts N-glycan profiles observed on human A1AT and IgG by MALDI MSI of spotted proteins. Glycoproteins were spotted (500 ng each) to a slide and imaged by MALDI FT-ICR for the detection of N-glycans on each protein. Percentage of each N-glycan specie was calculated by area under the peak divided by the total of all N-glycan peak areas. The proposed structures for all species comprising >1% of each protein's N-glycome are shown above, and the distinctness of N-glycan structures found between the two proteins is evident. The N-glycan compositions are represented by blue square for N-acetylglucosamine, green circle for mannose, red triangle for fucose, and yellow circle for galactose.

FIG. 16A through FIG. 16E depict the results of N-glycan detection by MALDI MSI of immunocaptured A1AT. (FIG. 16A) A1AT was spotted (1 μL) to slide without any blocking (above) and following blocking with 1% BSA (below). Imaging data shown is from the most abundant N-glycan observed on A1AT, which is not significantly detectable from the blocked sample. (FIG. 16B) A1AT was added in a 100 μL volume to a well containing both anti-A1AT and anti-IgG as adjacent spots. Red and blue circles were added for emphasis of antibody location. (FIG. 16C) A dilution series of A1AT standard solutions was added in triplicate to its antibody in 100 μL volumes. Imaging data was acquired at 250 μM and normalized to total ion count across the slide. N-glycan signal is seen at antibody spots with an observed increase in color intensity as more glycoprotein was added. (FIG. 16D) Quantifications of imaging data in FIG. 16C were performed calculating the area under the peak for each sample. Each data point represents the average +/−standard deviation of three samples. (FIG. 16E) N-glycan profiles of spotted versus captured A1AT were compared and showed strong agreement. Percentages of each N-glycan specie were calculated by area under the peak divided by the total of all N-glycan peak areas. The proposed structures for the most abundant N-glycans on A1AT are shown above. The N-glycan compositions are represented by blue square for N-acetylglucosamine, green circle for mannose, red triangle for fucose, and yellow circle for galactose.

FIG. 17A through FIG. 17E depict the results of side-by-side capture of A1AT and IgG from standard solutions and stock human serum. (FIG. 17A) Template of the 24-well module used on nitrocellulose-coated microscope slides. Within each well both anti-A1AT and anti-IgG were spotted adjacent at 200 ng per 1.5 μL spot. (FIG. 17B, FIG. 17C) Solutions containing a mixture of both A1AT and IgG standards were added in triplicate to wells containing the two antibodies. Red and blue circles are added to illustrate positions of anti-A1AT and anti-IgG, respectively. Imaging data was acquired at 250 μM and normalized to total ion count across the slide. An A1AT-associated N-glycan was observed localized to the left of each well, illustrating specific capture of this glycoprotein by anti-A1AT. An IgG-associated N-glycan was observed localized to the right of each well, illustrating specific capture of this glycoprotein by anti-IgG. (FIG. 17D, FIG. 17E) Stock human serum was diluted in PBS and added in triplicate to each well at only 1 μL of serum per 100 μL well. N-glycan signals from both A1AT and IgG were again detected localized to their respective antibodies. The N-glycan compositions are represented by blue square for N-acetylglucosamine, green circle for mannose, red triangle for fucose, and yellow circle for galactose.

FIG. 18A through FIG. 18D depict the results of detecting altered N-glycosylation in patient serum samples. (FIG. 18A) Stock human serum and serum pooled from 5 patients with cirrhosis were added in triplicate to wells containing both anti-A1AT and anti-IgG as shown previously. 1 μL of serum was diluted in 100 μL PBS for the addition to each well. Imaging data was acquired at 250 μM and normalized to total ion count across the slide. An IgG-associated N-glycan was observed to increase in the cirrhotic samples compared to the stock serum, which has been previously reported. (FIG. 18B, FIG. 18C) IgG N-glycan profiles in stock human serum and cirrhotic patient serum illustrating an increase in the nongalactosylated fucosylated biantennary N-glycan from FIG. 18A and a subsequent decrease in the galactosylated fucosylated biantennary N-glycans. Percentages of each N-glycan specie were calculated by area under the peak divided by the total of all N-glycan peak areas. (FIG. 18D) An A1AT-associated N-glycan is shown to illustrate that this glycoprotein was also specifically captured out of both stock and cirrhotic patient serum.

FIG. 19A through FIG. 19D depict the HPLC profiles of A1AT and IgG. (FIG. 19A, FIG. 19B) For orthogonal comparison of N-glycan profiles, A1AT and IgG were digested in-solution with PNGase F followed by HPLC analysis. (FIG. 19C, FIG. 19D) Percentage of each N-glycan specie was calculated by the peak area divided by the total of all N-glycan peak areas.

FIG. 20A through FIG. 20D depict the quantifications of N-glycan peaks observed in FIG. 17B through FIG. 17E, respectively. Area under peak values were obtained for each region. Bar represent the mean +/−standard deviation of 3 samples, and highlight that significant N-glycan signal was observed from each sample beyond that of the antibody background signal.

### DETAILED DESCRIPTION OF THE INVENTION

The present invention provides methods and compositions for glycan analysis of complex solutions, including proteins and cells in a biological sample. The method includes the preparation of substrates for the capture of proteins and cells for multiplexed analysis. Cells and proteins may be captured by antibody arrays, culture, or direct deposition. The invention further relates to the use of protein and cell glycan analysis in the diagnosis and screening of disease states and disease progression.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass non-limiting variations of ±40% or ±20% or ±10%, ±5%, ±1%, or ±0.1% from the specified value, as such variations are appropriate.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type, might be abnormal for a different cell or tissue type.

The terms “biomarker” and “marker” are used herein interchangeably. They refer to a substance that is a distinctive indicator of a biological process, biological event and/or pathologic condition.

The phrase “body sample” or “biological sample” is used herein in its broadest sense. A sample may be of any biological tissue or fluid from which biomarkers of the present invention may be assayed. Examples of such samples include but are not limited to blood, saliva, buccal smear, feces, lymph, urine, gynecological fluids, biopsies, amniotic fluid and smears. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art. Frequently, a sample will be a “clinical sample,” i.e., a sample derived from a patient. Such samples include, but are not limited to, bodily fluids which may or may not contain cells, e.g., blood (e.g., whole blood, serum or plasma), urine, saliva, tissue or fine needle biopsy samples, and archival samples with known diagnosis, treatment and/or outcome history. Biological or body samples may also include sections of tissues such as frozen sections taken for histological purposes. The sample also encompasses any material derived by processing a biological or body sample. Derived materials include, but are not limited to, cells (or their progeny) isolated from the sample, proteins or nucleic acid molecules extracted from the sample. Processing of a biological or body sample may involve one or more of: filtration, distillation, extraction, concentration, inactivation of interfering components, addition of reagents, and the like.

As used herein, the term “carbohydrate” is intended to include any of a class of aldehyde or ketone derivatives of polyhydric alcohols. Therefore, carbohydrates include starches, celluloses, gums and saccharides. Although, for illustration, the term “saccharide” or “glycan” is used elsewhere herein, this is not intended to be limiting. It is intended that the methods provided herein can be directed to any carbohydrate, and the use of a specific carbohydrate is not meant to be limiting to that carbohydrate only.

As used herein, the term “cell-surface glycoprotein” refers to a glycoprotein, at least a portion of which is present on the exterior surface of a cell. In some embodiments, a cell-surface glycoprotein is a protein that is positioned on the cell-surface such that at least one of the glycan structures is present on the exterior surface of the cell.

In the context of the present invention, the term “control,” when used to characterize a subject, refers, by way of non-limiting examples, to a subject that is healthy, to a patient that otherwise has not been diagnosed with a disease. The term “control sample” refers to one, or more than one, sample that has been obtained from a healthy subject or from a non-disease tissue such as normal colon.

The term “control or reference standard” describes a material comprising none, or a normal, low, or high level of one of more of the marker (or biomarker) expression products of one or more the markers (or biomarkers) of the invention, such that the control or reference standard may serve as a comparator against which a sample can be compared.

“Differentially increased levels” refers to biomarker levels which are at least 1%, 2%, 3%, 4%, 5%, 10% or more, for example, 5%, 10%, 20%, 30%, 40%, or 50%, 60%, 70%, 80%, 90% higher or more, and/or 0.5 fold, 1.1 fold, 1.2 fold, 1.4 fold, 1.6 fold, 1.8 fold higher or more, as compared with a control.

“Differentially decreased levels” refers to biomarker levels which are at least at least 1%, 2%, 3%, 4%, 5%, 10% or more, for example, 5%, 10%, 20%, 30%, 40%, or 50%, 60%, 70%, 80%, 90% lower or less, and/or 0.9 fold, 0.8 fold, 0.6 fold, 0.4 fold, 0.2 fold, 0.1 fold or less, as compared with a control.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease, or disorder, the frequency with which such a sign or symptom is experienced by a patient, or both, is reduced.

The terms “effective amount” and “pharmaceutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

As used herein “endogenous” refers to any material from or produced inside the organism, cell, tissue or system.

The term “expression” as used herein is defined as the transcription and/or translation of a particular nucleotide sequence driven by its promoter.

The “level” of one or more biomarkers means the absolute or relative amount or concentration of the biomarker in the sample. The term “level” also refers to the absolute or relative amount of glycosylation of the biomarker in the sample.

As is known in the art and used herein “glycans” are sugars (e.g., oligosaccharides and polysaccharides). Glycans can be monomers or polymers of sugar residues typically joined by glycosidic bonds also referred to herein as linkages. In some embodiments, the terms “glycan”, “oligosaccharide” and “polysaccharide” may be used to refer to the carbohydrate portion of a glycoconjugate (e.g., glycoprotein, glycolipid or proteoglycan). A glycan may include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′-sulfo N-acetylglucosamine, etc.). The term “glycan” includes homo and heteropolymers of sugar residues. The term “glycan” also encompasses a glycan component of a glycoconjugate (e.g., of a glycoprotein, glycolipid, proteoglycan, etc.). The term also encompasses free glycans, including glycans that have been cleaved or otherwise released from a glycoconjugate.

As used herein, the term “antibody array” refers to a tool used to identify glycans on proteins that interact with any of a number of different antibodies linked to the array substrate. In some embodiments, antibody arrays comprise a number of immobilized antibodies, referred to herein as “antibody spots”. In some embodiments, glycan arrays comprise at least 2, at least 5, at least 10, at least 20, at least 30, at least 40, at least 50, at least 60, at least 70, at least 80, at least 90, at least 100, at least 150, at least 350, at least 1000 or at least 1500 antibody spots. In some embodiments, antibody arrays may be customized to present a desired set of antibody spots.

The term “glycoconjugate”, as used herein, encompasses all molecules in which at least one sugar moiety is covalently linked to at least one other moiety. The term specifically encompasses all biomolecules with covalently attached sugar moieties, including for example N-linked glycoproteins, O-linked glycoproteins, glycolipids, proteoglycans, etc.

The term “glycoform”, is used herein to refer to a particular form of a glycoconjugate. That is, when the same backbone moiety (e.g., polypeptide, lipid, etc) that is part of a glycoconjugate has the potential to be linked to different glycans or sets of glycans, then each different version of the glycoconjugate (i.e., where the backbone is linked to a particular set of glycans) is referred to as a “glycoform.”

The term “glycosidase” as used herein refers to an agent that cleaves a covalent bond between sequential sugars in a glycan or between the sugar and the backbone moiety (e.g. between sugar and peptide backbone of glycoprotein). In some embodiments, a glycosidase is an enzyme. In certain embodiments, a glycosidase is a protein (e.g., a protein enzyme) comprising one or more polypeptide chains. In certain embodiments, a glycosidase is a chemical cleavage agent.

A “glycoprotein preparation”, as that term is used herein, refers to a set of individual glycoprotein molecules, each of which comprises a polypeptide having a particular amino acid sequence (which amino acid sequence includes at least one glycosylation site) and at least one glycan covalently attached to the at least one glycosylation site. Individual molecules of a particular glycoprotein within a glycoprotein preparation typically have identical amino acid sequences but may differ in the occupancy of the at least one glycosylation sites and/or in the identity of the glycans linked to the at least one glycosylation sites. That is, a glycoprotein preparation may contain only a single glycoform of a particular glycoprotein, but more typically contains a plurality of glycoforms. Different preparations of the same glycoprotein may differ in the identity of glycoforms present (e.g., a glycoform that is present in one preparation may be absent from another) and/or in the relative amounts of different glycoforms.

The term “lectin” as used herein encompasses any amino acid and peptide bond-based compound having specific binding affinity to carbohydrates. Typically it relates to non-antibody polypeptides found in nature featuring specific carbohydrate binding. The term “lectin” includes functional fragments and derivatives thereof, the latter terms being defined in analogy to the same terms used in the context of antibodies.

“Measuring” or “measurement,” or alternatively “detecting” or “detection,” means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

The term “N-glycan”, as used herein, refers to a polymer of sugars that has been released from a glycoconjugate but was formerly linked to the glycoconjugate via a nitrogen linkage. N-linked glycans are glycans that are linked to a glycoconjugate via a nitrogen linkage at asparagine residues within conserved protein structural motifs of N/X (any amino acid except proline)/S or T (serine or threonine). A diverse assortment of N-linked glycans exists, but is typically based on the common core pentasaccharide (Man)₃(GlcNAc)(GlcNAc).

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally occurring sequence.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand that are located 5′ to a reference point on the DNA are referred to as “upstream sequences”; sequences on the DNA strand which are 3′ to a reference point on the DNA are referred to as “downstream sequences.”

The term “O-glycan”, as used herein, refers to a polymer of sugars that has been released from a glycoconjugate but was formerly linked to the glycoconjugate via an oxygen linkage. O-linked glycans are glycans that are linked to a glycoconjugate via an oxygen linkage. O-linked glycans are typically attached to glycoproteins via N-acetyl-D-galactosamine (GalNAc) or via N-acetyl-D-glucosamine (GlcNAc) to the hydroxyl group of L-serine (Ser) or L-threonine (Thr). Some O-linked glycans also have modifications such as acetylation and sulfation. In some instances O-linked glycans are attached to glycoproteins via fucose or mannose to the hydroxyl group of L-serine (Ser) or L-threonine (Thr).

The term “pre-cancerous” or “pre-neoplastic” and equivalents thereof shall be taken to mean any cellular proliferative disorder that is undergoing malignant transformation. Examples of such conditions include, in the context of colorectal cellular proliferative disorders, cellular proliferative disorders with a high degree of dysplasia and the following classes of adenomas: Level 1: penetration of malignant glands through the muscularis mucosa into the submucosa, within the polyp head; Level 2: the same submucosal invasion, but present at the junction of the head to the stalk; Level 3: invasion of the stalk; and Level 4: invasion of the stalk's base at the connection to the colonic wall. In some instances, pre-neoplastic is used to describe a normal tissue that will form tumors.

As used herein, “predisposition” refers to the property of being susceptible to a cellular proliferative disorder. A subject having a predisposition to a cellular proliferative disorder has no cellular proliferative disorder, but is a subject having an increased likelihood of having a cellular proliferative disorder.

A “polynucleotide” means a single strand or parallel and anti-parallel strands of a nucleic acid. Thus, a polynucleotide may be either a single-stranded or a double-stranded nucleic acid. In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T.”

As used herein, the term “providing a prognosis” refers to providing a prediction of the probable course and outcome of colorectal cancer, including prediction of severity, duration, chances of recovery, etc. The methods can also be used to devise a suitable therapeutic plan, e.g., by indicating whether or not the condition is still at an early stage or if the condition has advanced to a stage where aggressive therapy would be ineffective.

A “reference level” of a biomarker means a level of the biomarker, for example level of a type of glycan that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype.

As used herein, the term “saccharide” refers to a polymer comprising one or more monosaccharide groups. Saccharides, therefore, include mono-, di-, tri- and polysaccharides (or glycans). Glycans can be branched or branched. Glycans can be found covalently linked to non-saccharide moieties, such as lipids or proteins (as a glycoconjugate). These covalent conjugates include glycoproteins, glycopeptides, peptidoglycans, proteoglycans, glycolipids and lipopolysaccharides. The use of any one of these terms also is not intended to be limiting as the description is provided for illustrative purposes. In addition to the glycans being found as part of a glycoconjugate, the glycans can also be in free form (i.e., separate from and not associated with another moiety).

By the term “specifically binds,” as used herein, is meant a molecule, such as an antibody, which recognizes and binds to another molecule or feature, but does not substantially recognize or bind other molecules or features in a sample.

“Standard control value” as used herein refers to a predetermined glycan level. The standard control value is suitable for the use of a method of the present invention, in order for comparing the amount of glycan of interest that is present in a sample. An established sample serving as a standard control provides an average amount of glycan of interest that is typical for an average, healthy person of reasonably matched background, e.g., gender, age, ethnicity, and medical history. A standard control value may vary depending on the biomarker of interest and the nature of the sample.

As used herein, the term “subject” refers to a human or another mammal (e.g., primate, dog, cat, goat, horse, pig, mouse, rat, rabbit, and the like. In many embodiments of the present invention, the subject is a human being. In such embodiments, the subject is often referred to as an “individual” or a “patient.” The terms “individual” and “patient” do not denote a particular age.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based in part on novel methods and devices that allow for the glycan analysis of hundreds and thousands of individual proteins and cells found in complex mixtures. The methods relate to the capture of specific proteins using an antibody array, treatment of the captured protein with highly active recombinant PNGase F, and glycan analysis of the specific captured proteins on a spot by spot basis by mass spectrometry. The methods also relate to the capture of cells using an antibody array or the deposition of cells onto a substrate, fixation and treatment of the cells, and glycan analysis of the cells by mass spectrometry. In certain instances, the methods are useful as a diagnostic platform for the detection of biomarkers associated with various diseases or disorders. Accordingly, the invention provides compositions and methods for glycan analysis for disease detection, diagnosis and prognosis, such as cancer.

In various embodiments, proteins and cells are profiled using mass spectroscopy. In another embodiment, proteins and cells are profiled using matrix-assisted laser desorption/ionization (MALDI). In another embodiment, proteins and cells are characterized using mass spectroscopy. In another embodiment, proteins and cells are characterized using MALDI Fourier transform ion cyclotron resonance (MALDI-FTICR) mass spectrometry. In another embodiment, proteins and cells are characterized using MALDI time of flight (MALDI-TOF) mass spectrometry.

### Glycan Analysis for Proteins in Antibody Arrays

The present invention provides in part antibody arrays that allow for the generation of structural glycan information for hundreds of individual glycoprotein targets. The antibody arrays utilize an efficient workflow that allows for the capture of specific proteins, treatment of captured protein with highly active recombinant PNGase F, and glycan analysis of the specific captured proteins on a spot by spot basis by mass spectrometry. The present invention also provides a platform for determining structural glycan information from as many proteins that can be captured on an antibody array.

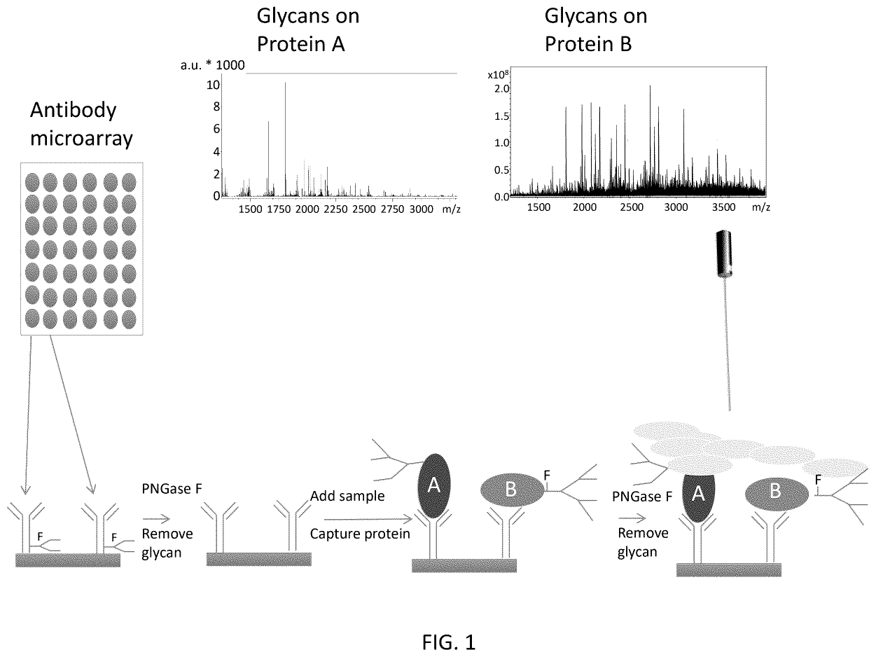

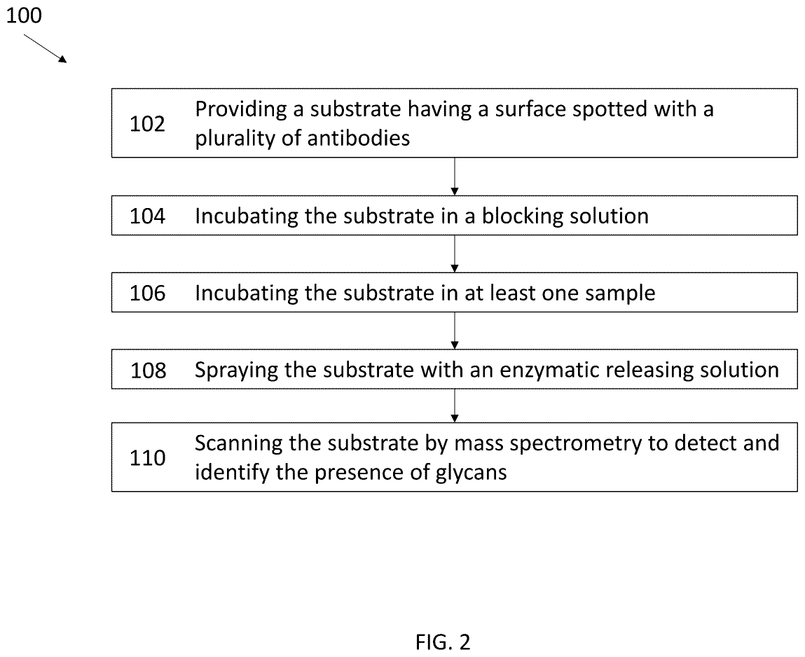

Referring now to FIG. 1 and FIG. 2, a schematic and a flowchart listing the steps of method 100 of an exemplary workflow for glycan analysis of proteins are depicted, respectively. Method 100 begins with step 102, wherein a substrate is provided having a surface spotted with a plurality of antibodies. The substrate can be any suitable substrate, such as a glass or plastic microscope slide or multiwell plate. In step 104, the substrate is incubated in a blocking solution. The blocking solution can be a serum solution, such as 1% BSA in PBS and detergent, and the incubation can be for one hour. In step 106, the substrate is incubated in at least one sample. In some embodiments, the sample is a protein sample. The protein sample can be incubated for 2 hours at room temperature in a humidity chamber. In step 108, the substrate is sprayed with an enzymatic releasing solution. The substrate can be sprayed with PNGase F and incubated overnight. In step 110, the substrate is scanned by mass spectrometry to detect and identify the presence of glycans. In certain embodiments, the substrate is washed between steps, such as with PBS baths, PBS and detergent baths, water baths, and combinations thereof.

The antibody array spotted on the substrate allows for capture and glycan analysis of hundreds to thousands of different proteins. Thus, the antibody arrays of the invention can comprise hundreds to thousands of different antibodies, each specific for one protein of interest. In certain embodiments, the antibody arrays of the invention specifically bind to a secreted protein of interest.

Spotting of antibodies can be achieved through any suitable technique, including but not limited to inkjet printing, fine print spotting, flow patterning on a functionalized substrate, contact printing on functionalized glass substrate, incubating on coated substrates (such as a nitrocellulose coating), or microprinting using epoxy-coated glass substrate or poly-amine glass substrate with printing needles or strips with very fine feature resolution.

The antibody arrays can be arranged in any desired grid or pattern. In certain embodiments, individual antibody spots are spaced laterally and longitudinally in an array of rows and/or columns. In one embodiment, individual antibody spots are regularly spaced at about 10-100 μm in separation. In one embodiment, the antibody arrays comprise about 10-1,000,000 individual antibody spots. In another embodiment, the antibody arrays comprise about 500-500,000 individual antibody spots. In another embodiment, the antibody arrays comprise about 100-100,000 individual antibody spots. In one embodiment, the antibody arrays comprise antibody spots at a density of about 200 antibody spots per cm² to about 20,000 antibody spots per cm². In various embodiments, arraying antibody spots can be aided with the use of one or more grids, such as a well slide module.

In certain embodiments, each spot comprises a single specific antibody. In another embodiment, each spot comprises 2, 3, 5, 10, or more different antibodies. In these embodiments, specific binding to a particular antibody within the feature can be determined by use of different detectable labels on a second set of capture agents, with each label corresponding to a particular antibody.

In various embodiments, the antibody arrays can include antibodies, antibody fragments, or combinations thereof. Such antibodies include polyclonal antibodies, monoclonal antibodies, Fab and single chain Fv (scFv) fragments thereof, bispecific antibodies, heteroconjugates, human and humanized antibodies. Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species.

Any suitable mass spectrometry imaging technique can be used. Non-limiting examples include matrix-assisted laser desorption/ionization imaging Fourier transform ion cyclotron resonance (MALDI-FTICR) mass spectrometry, matrix-assisted laser desorption/ionization time of flight (MALDI-TOF) mass spectrometry, scanning microprobe MALDI (SMALDI) mass spectrometry, infrared matrix assisted laser desorption electrospray ionization (MALD-ESI) mass spectrometry, surface-assisted laser desorption/ionization (SALDI) mass spectrometry, desorption electrospray ionization (DESI) mass spectrometry, secondary ion mass spectrometry (SIMS) mass spectrometry, easy ambient sonic spray ionization (EASI) mass spectrometry, and the like. In various embodiments, the substrate can be sprayed with a matrix solution just prior to mass spectrometry imaging. Any suitable solution can be used, including but not limited to 2,5-dihydroxybenzoic acid, α-cyano-4-hydroxycinnamic acid, sinapinic acid, 1,5-diaminonaphthalene, 9-aminoacridine, and the like.

While the methods describe the use of antibody arrays, it should be understood that any suitable capture molecule having an affinity to a protein of interest can be used to capture proteins as would be understood by those having skill in the art. For example, antibodies can be replaced or supplemented with one or more antigens, aptamers, affibodies, proteins, peptides, nucleic acids, carbon nanotubes, and fragments thereof. The capture molecules are also not limited to an array pattern, and can be provided in any shape or form desired.

### Glycan Analysis for Cell Populations

The present invention also provides in part methods that allow for the generation of structural glycan information from at least one population of cells. In some embodiments, the at least one population of cells is selectively captured by way of the antibody arrays described elsewhere herein. In some embodiments, the at least one population of cells is adhered to a substrate. In various embodiments, the methods utilize an efficient workflow that fixes and delipidates at least one population of cells, treats the at least one population of cells with highly active recombinant PNGase F, and performs glycan analysis on the at least one population of cells on a spot by spot basis by mass spectrometry.

Referring to FIG. 2, method 100 can be adapted for cell analysis, wherein the plurality of antibodies in step 102 can be selected for binding to a desired populations of cells, and the at least one sample in step 106 comprises at least one population of cells. For example, anti-CD4 antibodies can be used to capture CD4 positive T-cells from a sample and anti-CD8 antibodies can be used to capture CD8 positive T-cells from a sample. Following step 106 and prior to step 108, the at least one population of cells is fixed and rinsed. The fixing and rinsing agents can be any suitable agent, including but not limited to formalin, Carnoy's solution, paraformaldehyde, ethanol-based fixatives, polyethylene glycol-based fixatives, and the like. Rinsing aids in clearing away uncaptured particles as well as the selective removal of analytes to boost the detection of N-glycans. For example, the suitable agents fix and delipidate the cells without disrupting cell morphology.

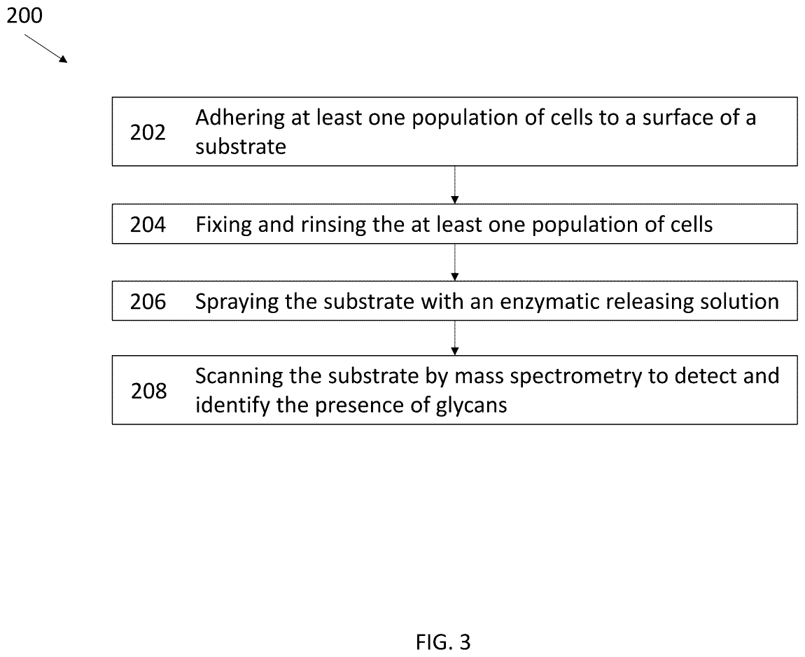

Referring now to FIG. 3, a flowchart listing the steps of method 200 of an exemplary workflow for glycan analysis of adhered populations of cells is depicted. Method 200 begins with step 202, wherein at least one population of cells is adhered to a surface of a substrate. The at least one population of cells can be adhered in any suitable manner, including but not limited to culturing, deposition of cell slurries, swabbing, smearing, centrifugation (e.g., Cytospin), and the like. The substrate can be any suitable substrate, such as a glass or plastic microscope slide or multiwell plate. In various embodiments, the substrate surface can be functionalized or coated to enhance cell adherence. For example, the substrate surface can include an indium tin oxide coating, a gelatin coating, a collagen coating, a poly-l-lysine coating, a poly-ornithine coating, an extracellular matrix coating, a protein coating (such as cadherins, immunoglobulins, selectins, mucins, integrins, and the like), surface ionization, and the like. In step 204, the at least one population of cells is fixed and rinsed. Suitable fixing and rinsing agents include but are not limited to formalin, Carnoy's solution, paraformaldehyde, ethanol-based fixatives, polyethylene glycol-based fixatives, and the like. In step 206, the substrate is sprayed with an enzymatic releasing solution. The substrate can be sprayed with PNGase F and incubated overnight. In step 208, the substrate is scanned by mass spectrometry to detect and identify the presence of glycans. In certain embodiments, the substrate is washed between steps, such as with PBS baths, PBS and detergent baths, water baths, and combinations thereof.

The at least one population of cells can be obtained from any desired source, including but not limited to blood, lymph, urine, gynecological fluids, tissue biopsies, amniotic fluid, bone marrow aspirates, and the like. The populations of cells can also be obtained from a source having a disease or disorder, including but not limited to: leukemia, bladder cancer, bone cancer, brain and spinal cord tumors, brain stem glioma, breast cancer, lung cancer, lymphoma, cervical cancer, colon cancer, colorectal cancer, esophageal cancer, gastrointestinal cancer, hepatocellular (liver) cancer, kidney (renal cell) cancer, melanoma, oral cancer, ovarian cancer, prostate cancer, and the like.

In various embodiments, the at least one population of cells can be cultured to expand the cell count, to adhere the at least one population of cells to the substrate surface, or both. As would be understood by those having skill in the art, direct deposition can be used for adherent and suspension cell lines, while culturing cells on the substrate surface is suitable for adherent cell lines. Typically, cells are grown in contact with a culture media. Culture media commonly comprises a basal medium, optionally supplemented with additional components. Basal medium is a medium that supplies essential sources of carbon and/or vitamins and/or minerals for the cells. The basal medium may or may not be free of protein. Media formulations that support the growth of cells include, but are not limited to, Minimum Essential Medium Eagle, ADC-1, LPM (bovine serum albumin-free), F10 (HAM), F12 (HAM), DCCM1, DCCM2, RPMI 1640, BGJ Medium (with and without Fitton-Jackson Modification), Basal Medium Eagle (BME-with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E-with Earle's salt base), Medium M199 (M199H-with Hank's salt base), Minimum Essential Medium Eagle (MEM-E-with Earle's salt base), Minimum Essential Medium Eagle (MEM-H-with Hank's salt base) and Minimum Essential Medium Eagle (MEM-NAA with nonessential amino acids), and the like. It is further recognized that additional components may be added to the culture medium. Such components include, but are not limited to, antibiotics, antimycotics, albumin, growth factors, amino acids, and other components known to the art for the culture of cells. Antibiotics which can be added into the medium include, but are not limited to, penicillin and streptomycin. However, the invention should in no way be construed to be limited to any one medium for culturing the cells of the invention. Rather, any media capable of supporting the cells of the invention in tissue culture may be used. Cells can be cultured at any desired density, including but not limited to about 100 cells/mL, 500 cells/mL, 1,000 cells/mL, 5,000 cells/mL, 10,000 cells/mL, 15,000 cells/mL, 20,000 cells/mL, and the like.

The at least one population of cells can be arranged in any desired grid or pattern. In certain embodiments, individual population of cells are arranged in regions spaced laterally and longitudinally in an array of rows and/or columns. In one embodiment, individual population of cells are arranged in regions that are regularly spaced at about 10-100 μm in separation. In one embodiment, the regions of cells comprise about 10-1,000,000 individual cell regions. In another embodiment, the regions of cells comprise about 500-500,000 individual cell regions. In another embodiment, the regions of cells comprise about 100-100,000 individual cell regions. In one embodiment, the cell regions comprise cells at a density of about 100 cells per region to about 100,000 cells per region. In various embodiments, arraying regions of cells can be aided with the use of one or more grids, such as a well slide module. culturing of cells is useful for expanding a cell or population of cells to generate a sufficient number of cells for a desired analytical method, for example genomic or expression analysis.

Any suitable mass spectrometry imaging technique can be used. Non-limiting examples include matrix-assisted laser desorption/ionization imaging Fourier transform ion cyclotron resonance (MALDI-FTICR) mass spectrometry, matrix-assisted laser desorption/ionization time of flight (MALDI-TOF) mass spectrometry, scanning microprobe MALDI (SMALDI) mass spectrometry, infrared matrix assisted laser desorption electrospray ionization (MALD-ESI) mass spectrometry, surface-assisted laser desorption/ionization (SALDI) mass spectrometry, desorption electrospray ionization (DESI) mass spectrometry, secondary ion mass spectrometry (SIMS) mass spectrometry, easy ambient sonic spray ionization (EASI) mass spectrometry, and the like. In various embodiments, the substrate can be sprayed with a matrix solution just prior to mass spectrometry imaging. Any suitable solution can be used, including but not limited to 2,5-dihydroxybenzoic acid, α-cyano-4-hydroxycinnamic acid, sinapinic acid, 1,5-diaminonaphthalene, 9-aminoacridine, and the like.

### Glycan Analysis

The analysis of N-linked glycans most often involves the analysis of either purified individual proteins or complex mixtures of proteins. Glycans play multi-faceted roles in many biological processes and aberrant glycosylation is associated with many diseases. Glycans are post-translation modifications of proteins that are involved in cell growth, cytokinesis, differentiation, transcription regulation, signal transduction, ligand-receptor binding, and interactions of cells with other cells, extracellular matrix, and bacterial and viral infection, among other functions. Glycan misregulations and structural changes occur in most of the diseases that affect the human.

The glycans detectable by the invention include straight chain and branched oligosaccharides as well as naturally occurring and synthetic glycans. For example, the glycan can be a glycoaminoacid, a glycopeptide, a glycolipid, a glycosaminoglycan (GAG), a glycoprotein, a whole cell, a cellular component, a glycoconjugate, a glycomimetic, a glycophospholipid anchor, glycosylphosphatidylinositol (GPI)-linked glycoconjugates, bacterial lipopolysaccharides and endotoxins. The glycans can also include N-glycans, B-glycans, glycolipids and glycoproteins.

In some instances, the glycans detectable by the invention include two or more sugar units. Any type of sugar unit can be present in the glycans of the invention, including, for example, allose, altrose, arabinose, glucose, galactose, gulose, fucose, fructose, idose, lyxose, mannose, ribose, talose, xylose, or other sugar units. Such sugar units can have a variety of modifications and substituents. For example, sugar units can have a variety of substituents in place of the hydroxy, carboxylate, and methylenehydroxy substituents. Thus, lower alkyl moieties can replace any of the hydrogen atoms from the hydroxy, carboxylic acid and methylenehydroxy substituents of the sugar units in the glycans of the invention. For example, amino acetyl can replace any of the hydroxy or hydrogen atoms from the hydroxy, carboxylic acid and methylenehydroxy substituents of the sugar units in the glycans of the invention.

In some embodiments, the methods of the present invention can include determining the glycoprofile of a glycoprotein. The properties can be determined by analyzing the glycans of the intact glycoprotein. Properties of the glycans which can be determined include: the mass of part or all of the saccharide structure, the charges of the chemical units of the saccharide, identities of the chemical units of the saccharide, confirmations of the chemical units of the saccharide, total charge of the saccharide, total number of sulfates of the saccharide, total number of acetates, total number of phosphates, presence and number of carboxylates, presence and number of aldehydes or ketones, dye-binding of the saccharide, compositional ratios of substituents of the saccharide, compositional ratios of anionic to neutral sugars, presence of uronic acid, enzymatic sensitivity, linkages between chemical units of the saccharide, charge, branch points, number of branches, number of chemical units in each branch, core structure of a branched or unbranched saccharide, the hydrophobicity and/or charge/charge density of each branch, absence or presence of GlcNAc and/or fucose in the core of a branched saccharide, number of mannose in an extended core of a branched saccharide, presence or absence or sialic acid on a branched chain of a saccharide, the presence or absence of galactose on a branched chain of a saccharide.

A property of a glycan can be identified by any means known in the art. For example, molecular weight can be determined by several methods including mass spectrometry. The use of mass spectrometry for determining the molecular weight of glycans is well known in the art. Mass spectrometry has been used as a powerful tool to characterize polymers such as glycans because of its accuracy in reporting the masses of fragments generated (e.g., by enzymatic cleavage), and also because only minute sample concentrations are required.

Any analytic method for analyzing the glycans so as to characterize them can be performed on any sample of glycans, such analytic methods include those described herein. As used herein, to “characterize” a glycan or other molecule means to obtain data that can be used to determine its identity, structure, composition or quantity. When the term is used in reference to a glycoconjugate, it can also include determining the glycosylation sites, the glycosylation site occupancy, the identity, structure, composition or quantity of the glycan and/or non-saccharide moiety of the glycoconjugate as well as the identity and quantity of the specific glycoform. These methods include, for example, mass spectrometry, nuclear magnetic resonance (NMR) (e.g., 2D-NMR), electrophoresis and chromatographic methods. Examples of mass spectrometric methods include fast atom bombardment mass spectrometry (FAB-MS), liquid chromatography mass spectrometry (LC-MS), liquid chromatography tandem mass spectrometry (LC-MS/MS), matrix-assisted laser desorption/ionization mass spectrometry (MALDI-MS), matrix-assisted laser desorption/ionization tandem mass spectrometry (MALDI-MS/MS), etc. NMR methods can include, for example, correlation spectroscopy (COSY), two-dimensional nuclear magnetic resonance spectroscopy (TOCSY), Nuclear Overhauser effect spectroscopy (NOESY). Electrophoresis can include, for example, capillary electrophoresis with laser induced fluorescence (CE-LIF), capillary gel electrophoresis (CGE), capillary zone electrophoresis (CZE), COSY, TOCSY, and NOESY.

Mass spectrometry imaging is a powerful tool that has been used to correlate various peptides, proteins, lipids and metabolites with their underlying histopathology in tissue sections. Taking advantage of the rapid advances in mass spectrometry, mass spectrometry imaging can push the limits of glycomics studies. Mass spectrometry imaging offers some advantages over the conventional methods that support its use as a complementary technique to lectin histochemistry. One significant advantage is that matrix-assisted laser desorption/ionization (MALDI) imaging combined with tandem mass spectrometry reveals detailed structural information about the glycans in a sample. A wide range of molecular weights can be detected by mass spectrometry imaging. Also, the high mass resolution allows distinguishing two peaks with close molecular weights, which subsequently improves the detection specificity. In addition, tens or even hundreds of glycans can be detected at femtomole levels in one single image, allowing detection of low concentrations of molecules. Therefore, MALDI imaging facilitates high-throughput analysis of tissue glycans. MALDI imaging can also be used for performing quantitative assays. Another significant advantage of MALDI imaging is that it has the capability of detecting an unknown compound without any a prior knowledge of the analytes. Therefore, this technique is particularly suitable for biomarker discovery research.

MALDI is a soft ionization mass spectrometric technique that is suitable for use in the analysis of biomolecules, such as proteins, peptides, sugars, and the like, which tend to be fragile and fragment when ionized by conventional ionization methods.

Generally, MALDI comprises a two-step process. In the first step, desorption is triggered by an ultraviolet (UV) laser beam. The matrix material absorbs the UV laser radiation, which leads to the ablation of an upper layer of the matrix material, thereby producing a hot plume. The hot plume contains many species: neutral and ionized matrix molecules, protonated and deprotonated matrix molecules, matrix clusters, and nanodroplets. In the second step, the analyte molecules are ionized, e.g., protonated or deprotonated, in the hot plume.

The matrix material comprises a crystallized molecule capable of absorbing the UV laser radiation. Common matrix materials include, but are not limited to, α-cyano-4-hydroxycinnamic acid, 2,5-dihydroxybenzoic acid, 2,5-dihydroxybenzoic acid/2-hydroxy-5-methoxybenzoic acid, 2,4,6-trihydroxyacetophenone, 6-aza-2-thiothymine, 3-hydroxypicolinic acid, 3-aminoquinoline, anthranilic acid, 5-chloro-2-mercaptobenzothiazole, 2,5-dihydroxyacetophenone, ferulic acid, and 2-(4-hydroxyphenylazo) benzoic acid. A solution of the matrix material is made in highly purified water and an organic solvent, such as acetonitrile or ethanol. In some embodiments, a small amount of trifluoroacetic acid (TFA) also can be added to the solution.

The matrix solution can then be mixed with the analyte, e.g., a protein sample. This solution is then deposited onto a MALDI plate, wherein the solvents vaporize leaving only the recrystallized matrix comprising the analyte molecules embedded in the MALDI crystals.

The property of the glycan that is detected by this method can also be any structural property of a glycan or unit. For instance, the property of the glycan can be the molecular mass or length of the glycan. In other embodiments the property can be the compositional ratios of substituents or units, type of basic building block of a polysaccharide, hydrophobicity, enzymatic sensitivity, hydrophilicity, secondary structure and conformation (i.e., position of helices), spatial distribution of substituents, linkages between chemical units, number of branch points, core structure of a branched polysaccharide, ratio of one set of modifications to another set of modifications (i.e., relative amounts of sulfation, acetylation or phosphorylation at the position for each), and binding sites for proteins.

Methods of identifying other types of properties are easily identifiable to those of skill in the art and generally can depend on the type of property and the type of glycan; such methods include, but are not limited to capillary electrophoresis (CE), NMR, mass spectrometry (both MALDI and ESI), and high performance liquid chromatography (HPLC) with fluorescence detection. For example, hydrophobicity can be determined using reverse-phase high-pressure liquid chromatography (RP-HPLC). Enzymatic sensitivity can be identified by exposing the glycan to an enzyme and determining a number of fragments present after such exposure. The chirality can be determined using circular dichroism. Protein binding can be determined by mass spectrometry, isothermal calorimetry and NMR. Linkages can be determined using NMR and/or capillary electrophoresis. Enzymatic modification (not degradation) can be determined in a similar manner as enzymatic degradation, i.e., by exposing a substrate to the enzyme and using MALDI-MS to determine if the substrate is modified. For example, a sulfotransferase can transfer a sulfate group to an oligosaccharide chain having a concomitant increase of 80 Da. Conformation can be determined by modeling and nuclear magnetic resonance (NMR). The relative amounts of sulfation can be determined by compositional analysis or approximately determined by Raman spectroscopy.

In accordance with an embodiment, the present invention provides a mass spectroscopy imaging technique that has been developed for profiling of glycans from proteins captured by an antibody array. A releasing agent can be sprayed over the captured proteins to release glycans. Common enzymatic releasing agents include, but are not limited to, trypsin, Endoglycosidase H (Endo H), Endoglycosidase F (EndoF), N-Glycanase F (PNGase F), PNGase A, O-glycanase, and/or one or more proteases (e.g., trypsin, or LysC), or chemically (e.g., using anhydrous hydrazine (N) or reductive or non-reductive beta-elimination (O)).

In other embodiments, the glycans can be modified to improve ionization of the glycans, particularly when MALDI-MS is used for analysis. Such modifications include permethylation. Another method to increase glycan ionization is to conjugate the glycan to a hydrophobic chemical (such as AA, AB labeling) for MS or liquid chromatographic detection. In other embodiments, spot methods can be employed to improve signal intensity.

Practical m/z ranges comprising most of the important signals, as observed by the present invention, may be more limited than these. Practical ranges includes lower limit of about m/z 400, about m/z 500, about m/z 600, and about m/z 700; and upper limits of about m/z 4000, about m/z 3500 (especially for negative ion mode), about m/z 3000 (especially for negative ion mode), and in particular at least about m/z 2500 (negative or positive ion mode) and for positive ion mode to about m/z 2000 (for positive ion mode analysis). The ranges depend on the sizes of the sample glycans, samples with high branching or polysaccharide content or high sialylation levels can be analyzed in ranges containing higher upper limits as described for negative ion mode. The limits can be combined to form ranges of maximum and minimum sizes or lowest lower limit with lowest higher limit, and the other limits analogously in order of increasing size.

Methods of the present disclosure can be applied to protein samples obtained from a wide variety of biological samples. A biological sample may undergo one or more analysis and/or purification steps prior to or after being analyzed according to the present disclosure. For example, in some embodiments, glycans in a biological sample are labeled with one or more detectable markers or other agents that may facilitate analysis by, for example, mass spectrometry or NMR. Any of a variety of separation and/or isolation steps may be applied to a biological sample in accordance with the present disclosure.

### Method of Diagnosis

Alterations in glycosylation have been associated with a number of diseases. Generally, these changes are observed through glycan analysis of complex protein mixtures or through the analysis of a few specific individual proteins. In various embodiments, the present invention also provides methods for diagnosing a disease state or disorder state or the progression of a disease state or disorder state by detecting one or more specific glycans whose presence or level (whether absolute or relative) may be correlated with a particular disease state (including susceptibility to a particular disease) and/or the change in the concentration of such glycans over time.

The detected glycans and detected changes in glycosylation can be used to towards detecting, treating and/or preventing a variety of early stage diseases and/or cancers. In some embodiments, the presence of such glycans is indicative of the presence of cancer and can provide information on the prognosis of such a disease, for example, whether the disease is in remission or is becoming more aggressive. Patients with familial history of cancer, and hence a heightened risk of developing the disease, can be tested regularly to monitor their propensity for disease.

In some embodiments, the methods of the present invention provide a method of diagnosing a disease or condition in a subject comprising the steps of detecting the glycans present in a biological sample from a subject, establishing a glycan profile for the subject, comparing the glycan profile from the subject to glycan profile from a normal sample or diseased sample, and determining whether the subject has the disease or condition, wherein the glycans are detected using the presently disclosed methods described elsewhere herein.

For example, in some embodiments, the methods provide an antibody array for capturing proteins of interest having glycosylation patterns indicative of hepatocellular carcinoma (HCC). The antibody array can thereby include, but is not limited to, antibodies that specifically bind one or more of: A1AT, fetuin-A, hemopexin, Apo-J, LMW Kininogen, HMW Kininogen, apo-H, transferrin, IgG, IgM, IgA, fibronectin, laminin, ceruloplasmin, fibulin, angiotensinogen, Fibrillin-1, TIMP1, thrombospondin 1, galectin-3 binding protein, complement C1 R, clusterin, galectin 1, alpha-2-macroglobulin, Vitamin D binding protein, histidine rich glycoprotein, histidine rich glycoprotein, CD109, CEA, Cathepsin, AFP, and GP73. Capture of proteins from a subject's biological sample and subsequent glycan analysis can determine whether the subject has HCC and the current stage of HCC progression.

The diagnosis can be carried out in a person with or thought to have a disease or condition. The diagnosis can also be carried out in a person thought to be at risk for a disease or condition. “A person at risk” is one that has either a genetic predisposition to have the disease or condition or is one that has been exposed to a factor that could increase his/her risk of developing the disease or condition.

Detection of cancers at an early stage is crucial for its efficient treatment. Despite advances in diagnostic technologies, many cases of cancer are not diagnosed and treated until the malignant cells have invaded the surrounding tissue or metastasized throughout the body. Although current diagnostic approaches have significantly contributed to the detection of cancer, they still present problems in sensitivity and specificity.

In accordance with one or more embodiments of the present invention, it will be understood that the types of cancer diagnosis which may be made, using the methods provided herein, is not necessarily limited. For purposes herein, the cancer can be any cancer. As used herein, the term “cancer” is meant any malignant growth or tumor caused by abnormal and uncontrolled cell division that may spread to other parts of the body through the lymphatic system or the blood stream.

The cancer can be a metastatic cancer or a non-metastatic (e.g., localized) cancer. As used herein, the term “metastatic cancer” refers to a cancer in which cells of the cancer have metastasized, e.g., the cancer is characterized by metastasis of a cancer cells. The metastasis can be regional metastasis or distant metastasis, as described herein.

In accordance with an embodiment, the present invention provides a use of a glycan profile prepared using the method disclosed herein to diagnose a disease or condition in a subject, comprising comparing the glycan profile from a subject to a glycan profile from a normal sample, or diseased sample, and determining whether the sample of the subject has the disease or condition.

In accordance with the inventive methods, the terms “cancers” or “tumors” also include but are not limited to adrenal gland cancer, biliary tract cancer; bladder cancer, brain cancer; breast cancer; cervical cancer; choriocarcinoma; colon cancer; endometrial cancer; esophageal cancer; extrahepatic bile duct cancer; gastric cancer; head and neck cancer; intraepithelial neoplasms; kidney cancer; leukemia; lymphomas; liver cancer; lung cancer (e.g. small cell and non-small cell); melanoma; multiple myeloma; neuroblastomas; oral cancer; ovarian cancer; pancreas cancer; prostate cancer; rectal cancer; sarcomas; skin cancer; small intestine cancer; testicular cancer; thyroid cancer; uterine cancer; urethral cancer and renal cancer, as well as other carcinomas and sarcomas.

An extensive listing of cancer types includes but is not limited to acute lymphoblastic leukemia (adult), acute lymphoblastic leukemia (childhood), acute myeloid leukemia (adult), acute myeloid leukemia (childhood), adrenocortical carcinoma, adrenocortical carcinoma (childhood), AIDS-related cancers, AIDS-related lymphoma, anal cancer, astrocytoma (childhood cerebellar), astrocytoma (childhood cerebral), basal cell carcinoma, bile duct cancer (extrahepatic), bladder cancer, bladder cancer (childhood), bone cancer (osteosarcoma/malignant fibrous histiocytoma), brain stem glioma (childhood), brain tumor (adult), brain tumor—brain stem glioma (childhood), brain tumor—cerebellar astrocytoma (childhood), brain tumor—cerebral astrocytoma/malignant glioma (childhood), brain tumor—ependymoma (childhood), brain tumor—medulloblastoma (childhood), brain tumor—supratentorial primitive neuroectodermal tumors (childhood), brain tumor—visual pathway and hypothalamic glioma (childhood), breast cancer (female, male, childhood), bronchial adenomas/carcinoids (childhood), Burkitt's lymphoma, carcinoid tumor (childhood), carcinoid tumor (gastrointestinal), carcinoma of unknown primary site (adult and childhood), central nervous system lymphoma (primary), cerebellar astrocytoma (childhood), cerebral astrocytoma/malignant glioma (childhood), cervical cancer, chronic lymphocytic leukemia, chronic myelogenous leukemia, chronic myeloproliferative disorders, colon cancer, colorectal cancer (childhood), cutaneous t-cell lymphoma, endometrial cancer, ependymoma (childhood), esophageal cancer, esophageal cancer (childhood), Ewing's family of tumors, extracranial germ cell tumor (childhood), extragonadal germ cell tumor, extrahepatic bile duct cancer, eye cancer (intraocular melanoma and retinoblastoma), gallbladder cancer, gastric (stomach) cancer, gastric (stomach) cancer (childhood), gastrointestinal carcinoid tumor, gastrointestinal stromal tumor (gist), germ cell tumor (extracranial (childhood), extragonadal, ovarian), gestational trophoblastic tumor, glioma (adult), glioma (childhood: brain stem, cerebral astrocytoma, visual pathway and hypothalamic), hairy cell leukemia, head and neck cancer, hepatocellular (liver) cancer (adult primary and childhood primary), Hodgkin's lymphoma (adult and childhood), Hodgkin's lymphoma during pregnancy, hypopharyngeal cancer, hypothalamic and visual pathway glioma (childhood), intraocular melanoma, islet cell carcinoma (endocrine pancreas), Kaposi's sarcoma, kidney (renal cell) cancer, kidney cancer (childhood), laryngeal cancer, laryngeal cancer (childhood), leukemia—acute lymphoblastic (adult and childhood), leukemia, acute myeloid (adult and childhood), leukemia—chronic lymphocytic, leukemia—chronic myelogenous, leukemia—hairy cell, lip and oral cavity cancer, liver cancer (adult primary and childhood primary), lung cancer—non-small cell, lung cancer—small cell, lymphoma—AIDS-related, lymphoma—Burkitt's, lymphoma—cutaneous t-cell, lymphoma—Hodgkin's (adult, childhood and during pregnancy), lymphoma—non-Hodgkin's (adult, childhood and during pregnancy), lymphoma—primary central nervous system, macroglobulinemia—Waldenström's, malignant fibrous histiocytoma of bone/osteosarcoma, medulloblastoma (childhood), melanoma, melanoma—intraocular (eye), Merkel cell carcinoma, mesothelioma (adult) malignant, mesothelioma (childhood), metastatic squamous neck cancer with occult primary, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia, chronic, myeloid leukemia (adult and childhood) acute, myeloma—multiple, myeloproliferative disorders—chronic, nasal cavity and paranasal sinus cancer, nasopharyngeal cancer, nasopharyngeal cancer (childhood), neuroblastoma, non-small cell lung cancer, oral cancer (childhood), oral cavity and lip cancer, oropharyngeal cancer, osteosarcoma/malignant fibrous histiocytoma of bone, ovarian cancer (childhood), ovarian epithelial cancer, ovarian germ cell tumor, ovarian low malignant potential tumor, pancreatic cancer, pancreatic cancer (childhood), pancreatic cancer—islet cell, paranasal sinus and nasal cavity cancer, parathyroid cancer, penile cancer, pheochromocytoma, pineoblastoma and supratentorial primitive neuroectodermal tumors (childhood), pituitary tumor, plasma cell neoplasm/multiple myeloma, pleuropulmonary blastoma, pregnancy and breast cancer, primary central nervous system lymphoma, prostate cancer, rectal cancer, renal cell (kidney) cancer, renal cell (kidney) cancer (childhood), renal pelvis and ureter—transitional cell cancer, retinoblastoma, rhabdomyosarcoma (childhood), salivary gland cancer, salivary gland cancer (childhood), sarcoma—Ewing's family of tumors, sarcoma—Kaposi's, sarcoma—soft tissue (adult and childhood), sarcoma—uterine, Sézary syndrome, skin cancer (non-melanoma), skin cancer (childhood), skin cancer (melanoma), skin carcinoma—Merkel cell, small cell lung cancer, small intestine cancer, soft tissue sarcoma (adult and childhood), squamous cell carcinoma, squamous neck cancer with occult primary—metastatic, stomach (gastric) cancer, stomach (gastric) cancer (childhood), supratentorial primitive neuroectodermal tumors (childhood), testicular cancer, thymoma (childhood), thymoma and thymic carcinoma, thyroid cancer, thyroid cancer (childhood), transitional cell cancer of the renal pelvis and ureter, trophoblastic tumor, gestational, ureter and renal pelvis—transitional cell cancer, urethral cancer, uterine cancer—endometrial, uterine sarcoma, vaginal cancer, visual pathway and hypothalamic glioma (childhood), vulvar cancer, Waldenström's macroglobulinemia, and Wilms' tumor.

### Glycan Carrier Proteins Attached to Specific Glycans

The invention is further directed to methods of identifying glycans attached to any of the proteins captured by the antibody arrays of the invention, such as from integral (cell bound/transmembrane) cancer tissue or cell released proteins and assigning the glycan structures with specific carrier proteins, by specific purification of the protein, e.g. by affinity methods such as immunoprecipitation or by sequencing, by mass spectrometric sequencing, glycopeptides including sequencing and recognizing peptides and thus captured proteins linked to the glycans.

In some embodiments, the determined glycosylation marker of cancer can be used for identifying and isolating one or more glycoprotein biomarkers, i.e. glycoproteins that are specific for particular type of cancer. The glycoprotein biomarker of the disease carries the glycosylation marker of cancer. The isolation of the glycoprotein biomarkers of the cancer can be carried out using lectins or monoclonal antibodies.

The glycosylation of a protein may be indicative of a normal or a disease state. Therefore, methods are provided for diagnostic purposes based on the analysis of the glycosylation of a captured protein or set of captured proteins, such as the total glycome. The methods provided herein can be used for the diagnosis of any disease or condition that is caused or results in changes in a particular protein glycosylation or pattern of glycosylation. These patterns can then be compared to “normal” and/or “diseased” patterns to develop a diagnosis, and treatment for a subject. For example, the methods provided can be used in the diagnosis of cancer, inflammatory disease, benign prostatic hyperplasia (BPH), etc.

The diagnosis can be carried out in a person with or thought to have a disease or condition. The diagnosis can also be carried out in a person thought to be at risk for a disease or condition. “A person at risk” is one that has either a genetic predisposition to have the disease or condition or is one that has been exposed to a factor that could increase his/her risk of developing the disease or condition.

The present invention provides glycosylation markers associated with cancer. In one embodiment, the glycosylation marker is an organic biomolecule which is differentially present in a sample taken from an individual of one phenotypic status (e.g., having a disease) as compared with an individual of another phenotypic status (e.g., not having the disease). A biomarker is differentially present between the two individuals if the mean or median expression level, including glycosylation level, of the biomarker in the different individuals is calculated to be statistically significant. Biomarkers, alone or in combination, provide measures of relative risk that an individual belongs to one phenotypic status or another. Therefore, they are useful as markers for diagnosis of disease, the severity of disease, therapeutic effectiveness of a drug, and drug toxicity.

In one embodiment, the method of the invention is carried out by obtaining a set of measured values for a plurality of biomarkers from a biological sample derived from a test individual, obtaining a set of measured values for a plurality of biomarkers from a biological sample derived from a control individual, comparing the measured values for each biomarker between the test and control sample, and identifying biomarkers which are significantly different between the test value and the control value, also referred to as a reference value.

The process of comparing a measured value and a reference value can be carried out in any convenient manner appropriate to the type of measured value and reference value for the biomarker of the invention. For example, “measuring” can be performed using quantitative or qualitative measurement techniques, and the mode of comparing a measured value and a reference value can vary depending on the measurement technology employed. For example, when a qualitative colorimetric assay is used to measure biomarker levels, the levels may be compared by visually comparing the intensity of the colored reaction product, or by comparing data from densitometric or spectrometric measurements of the colored reaction product (e.g., comparing numerical data or graphical data, such as bar charts, derived from the measuring device). However, it is expected that the measured values used in the methods of the invention will most commonly be quantitative values (e.g., quantitative measurements of concentration). In other examples, measured values are qualitative. As with qualitative measurements, the comparison can be made by inspecting the numerical data, or by inspecting representations of the data (e.g., inspecting graphical representations such as bar or line graphs).

A measured value is generally considered to be substantially equal to or greater than a reference value if it is at least about 95% of the value of the reference value. A measured value is considered less than a reference value if the measured value is less than about 95% of the reference value. A measured value is considered more than a reference value if the measured value is at least more than about 5% greater than the reference value.

The process of comparing may be manual (such as visual inspection by the practitioner of the method) or it may be automated. For example, an assay device (such as a luminometer for measuring chemiluminescent signals) may include circuitry and software enabling it to compare a measured value with a reference value for a desired biomarker. Alternately, a separate device (e.g., a digital computer) may be used to compare the measured value(s) and the reference value(s). Automated devices for comparison may include stored reference values for the biomarker(s) being measured, or they may compare the measured value(s) with reference values that are derived from contemporaneously measured reference samples.

The above method for screening biomarkers can find biomarkers that are differentially glycosylated in cancer as well as at various dysplasic stages of the tissue which progresses to cancer. The screened biomarker can be used for cancer screening, risk-assessment, prognosis, disease identification, the diagnosis of disease stages, and the selection of therapeutic targets.

According to the method of the present invention, the progression of cancer at various stages or phases can be diagnosed by determining the glycosylation stage of one or more biomarkers obtained from a sample. By comparing the glycosylation stage of a biomarker from a sample at each stage of cancer with the glycosylation stage of one or more biomarkers isolated from a sample in which there is no cell proliferative disorder of tissue, a specific stage of cancer in the sample can be detected. In one embodiment, the glycosylation stage may be hyperglycosylation. In another embodiment, the glycosylation stage may be hypoglycosylation.

### Biological Samples

The present invention is directed to analysis of protein solutions derived biological samples. In some embodiments, the biological samples of interest are sourced from cancerous tissue, such as benign and/or malignant cancer tissue or tumors.

In one embodiment, the tissue is human tissue or tissue part such as liquid tissue, cell and/or solid polycellular tumors, and in another embodiment a solid human tissue that can be processed into a tissue solution. The tissues can be used for the analysis and/or targeting specific glycan marker structures from the tissues, including intracellularly and extracellularly, such as cell surface associated, localized markers. The individual cell type cancers or tumors include blood derived tumors such as leukemias and lymphomas, while solid tumors include solid tumors derived from solid tissues such as gastrointestinal tract tissues, other internal organs such as liver, kidneys, spleen, pancreas, lungs, gonads and associated organs including ovary, testicle, and prostate. The invention further reveals markers from individually or multicellularly presented cancer cells. The cancer cells include metastatic cells released from tumors/cancer and blood cell derived cancers, such as leukemias and/or lymphomas. Metastasis from solid tissue tumors forms a separate class of cancer samples with specific characteristics.

The cancer tissue materials to be analyzed according to the invention are in the invention also referred as tissue materials or simply as cells, because all tissues comprise cells, however the invention can be directed to unicellularly and/or multicellularly expressed cancer cells and/or solid tumors as separate characteristics. The invention further reveals normal tissue materials to be compared with the cancer materials. The invention is specifically directed to methods according to the invention for revealing status of transformed tissue or suspected cancer sample when expression of specific structure of a signal correlated with it is compared to a expression level estimated to correspond to expression in normal tissue or compared with the expression level in an standard sample from the same tissue, such as a tissue sample from healthy part of the same tissue from the same patient.

The invention is in some embodiments directed to analysis of the marker structures and/or glycan profiles from both cancer tissue and corresponding normal tissue of the same patient because part of the glycosylations includes individual changes for example related to rare glycosylation related diseases such as congenital disorders in glycosylation (of glycoproteins/carbohydrates) and/or glycan storage diseases. The invention is furthermore directed to method of verifying analyzing importance and/or change of a specific structure/structure group or glycan group in glycome in specific cancer and/or a subtype of a cancer optionally with a specific status (e.g. primary cancer, metastatic, benign transformation related to a cancer) by methods according to the present invention.

### Diagnostic Tests

In one embodiment, diagnostic tests that use the biomarkers of the invention (e.g., glycans) exhibit a sensitivity and specificity of at least 75%, at least 80%, at least 85%, at least 90%, at least 95%, at least 98% and about 100%. In some instances, screening tools of the present invention exhibit a high sensitivity of at least 75%, at least 80%, at least 85%, at least 90%, at least 95%, at least 98% and about 100%.

In one embodiment, the sensitivity is from about 75% to about 99%, or from about 80% to about 90%, or from about 80% to about 85%. In other embodiments, the specificity is from about 75% to about 99%, or from about 80% to about 90%, or from about 80% to about 85%.

In another embodiment, the present invention enables the screening of at-risk populations for the early detection of cancers, for example pancreatic cancer. Furthermore, in certain aspects, the present invention enables the differentiation of neoplastic (e.g. malignant) from benign (i.e. non-cancerous) cellular proliferative disorders.

The prognostic methods can be used to identify patients with cancer or at risk of cancer. Such patients can be offered additional appropriate therapeutic or preventative options, including endoscopic polypectomy or resection, and when indicated, surgical procedures, chemotherapy, radiation, biological response modifiers, or other therapies. Such patients may also receive recommendations for further diagnostic or monitoring procedures, including but not limited to increased frequency of colonoscopy, virtual colonoscopy, video capsule endoscopy, PET-CT, molecular imaging, or other imaging techniques.

Following the diagnosis of a subject according to the methods of the invention, the subject diagnosed with cancer or at risk for having a proliferative disease, such as cancer can be treated against the disease. The subject can be subjected to the diagnostic tests throughout treatment to monitor progress and effectiveness.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out exemplary embodiments of the present invention, and are not to be construed as limiting in any way the remainder of the disclosure.

### Example 1: Glycan Analysis Using MALDI Imaging Mass Spectrometry (MALDI-IMS)

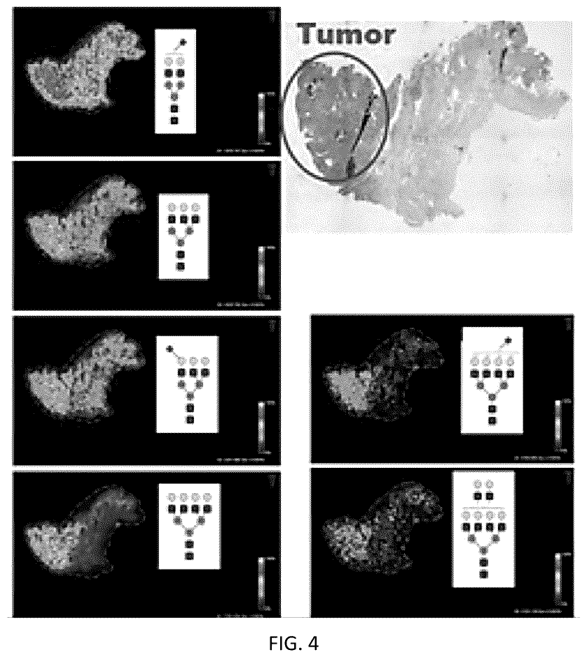

The following study provides a new method for glycan analysis of tissue using MALDI-IMS. The method bypasses the need for microdissection and solubilization of tissue proteins prior to analysis. MALDI-IMS has been extensively reviewed and generally employs a scheme where a matrix solution (such as sinapinic acid or dihydroxybenzoic acid (DHB)) is directly deposited onto tissue sections, and soluble molecules are extracted from the tissue and cocrystallized with the matrix. The matrix is applied across the tissue section such that desorption can be targeted to specific “points” in a grid pattern and the data rasterized. The resulting spectra can then be used to generate two-dimensional molecular maps of hundreds of analytes directly from the surface of a tissue section. These molecular maps display the relative abundance and spatial distribution of these molecules. Thus, MALDI tissue profiling has the power to link the molecular detail of mass spectrometry with molecular histology, generating mass spectra correlated to known locations within a thin tissue section. Most applications of MALDI-IMS have focused on profiling of proteins, lipids, and drug metabolites, but not glycans. Here, a molecular coating of recombinant endoglycosidase PNGase F is sprayed on formalin-fixed paraffin embedded (FFPE) tissues and used to remove N-linked glycan from tissue. FIG. 4 presents an example of this methodology for HCC tissue and adjacent tissue, showing just 6 of the N-linked glycans identified (out of over 100). It is clear to see that specific glycan patterns that are associated with the malignant tissue are observable. Importantly, they are localized within the tissue and can impart a level of localization of each glycan to particular area in the tissue.

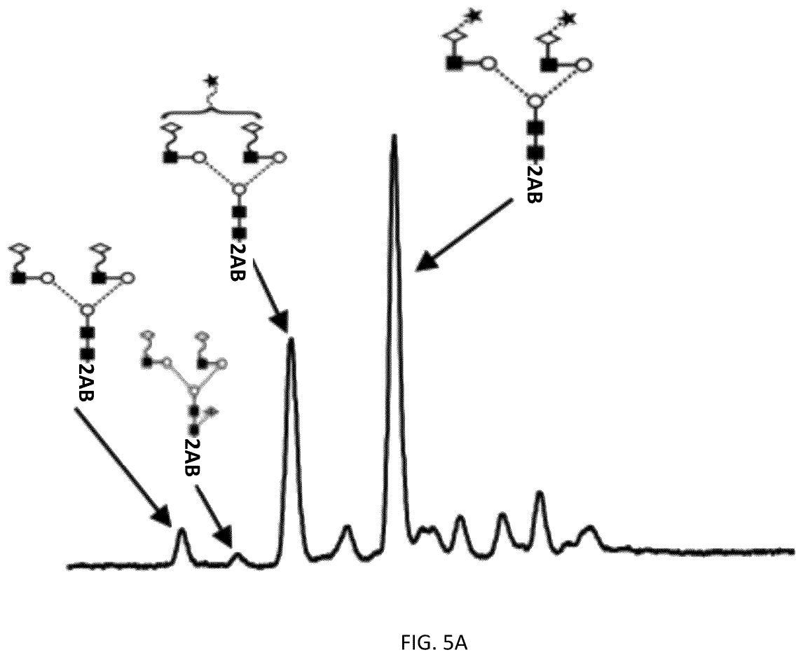

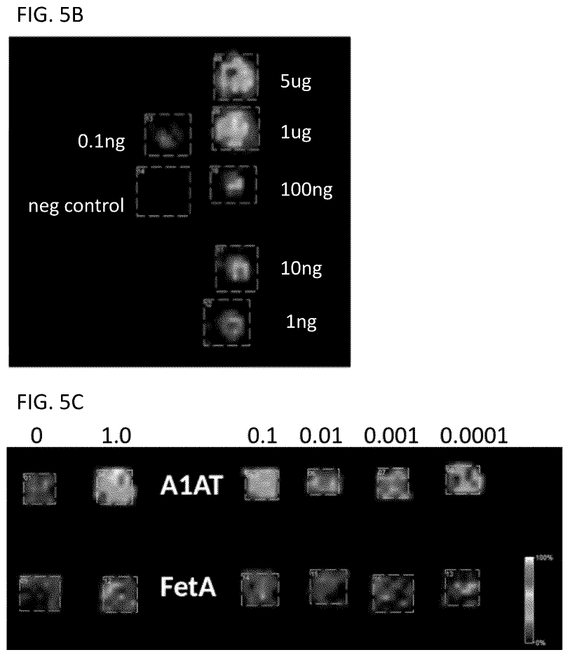

Spatially localization of glycan on tissue enables the identification of glycan captured onto specific areas of a slide. The basic method is shown in FIG. 1. The key feature of this methodology is the antibody capture of specific proteins and the analysis of N-linked glycan (after enzymatic release with PNGase F) by MALDI-FTICR MS. In an effort to determine how easily pure protein spotted onto a glass slide could be detected, the absolute sensitivity of the system was determined by spotting pure glycoprotein onto a glass slide followed by MALDI-FTICR MS. FIG. 5A through FIG. 5C presents preliminary evidence regarding the sensitivity of glycan detection. Spotted alpha 1 anti-trypsin (A1AT) was utilized, as this is a glycoprotein with significant structural glycan information. FIG. 5A shows the HPLC glycan profile of A1AT highlighting the presence of bi-antennary glycan with 0, 1, or 2 sialic acids. In addition, there is a small degree of core fucosylated biantennary glycan. FIG. 5B shows the detection of this minor peak, the core fucosylated bi-antennary glycan (m/z=1809.639), after directly spotting onto glass slides. Importantly, at 0.1 ng of spotted protein, this glycan can be detected. It is noted that the di-sialylated bi-antennary peak, the most abundant glycan on A1AT, was detected at a ratio of ~12:1 to the core fucosylated bi-antennary glycan.

FIG. 5C highlights the ability to detect glycans on A1AT captured using an antibody. Briefly, antibody to A1AT was spotted at a concentration of 300 ng per spot, the slides washed and blocked overnight with 2% BSA in PBS. As a specificity control, antibody to human fetuin-A was also spotted at a concentration of 300 ng/spot. The slide was sprayed with PNGase F to remove the inherent glycosylation of the capture antibody and washed. Spots were covered with varying amount of A1AT (from 1.0 μg to 0.0001 μg) and allowed to incubate for 1 hour after which the slide was washed with PBS, sprayed with PNGAse F, incubated again for 1 hour, sprayed with matrix and analyzed by MALDI-FTICR MS. As FIG. 5C shows, the core fucosylated bi-antennary glycan (1809.639) can be detected from 0.1 ng of captured A1AT. When no A1AT was added (labeled 0) or when A1AT was spotted onto an area where antibody to fetuin A was coated (a non specific protein), very weak signal is seen. As with the pure protein, the mono and di-sialylated biantennary glycan were observed at ratios of 12:1 to the core fucosylated bi-antennary peak (not shown). It is also noted that the signal to noise ratio for even the lowest level of protein is excellent with MS intensities over 100,000 for the bi-antennary glycan and essentially zero for the negative control spot. This spot contained no protein, while “negative control” spots will contain de-glycosylated protein.

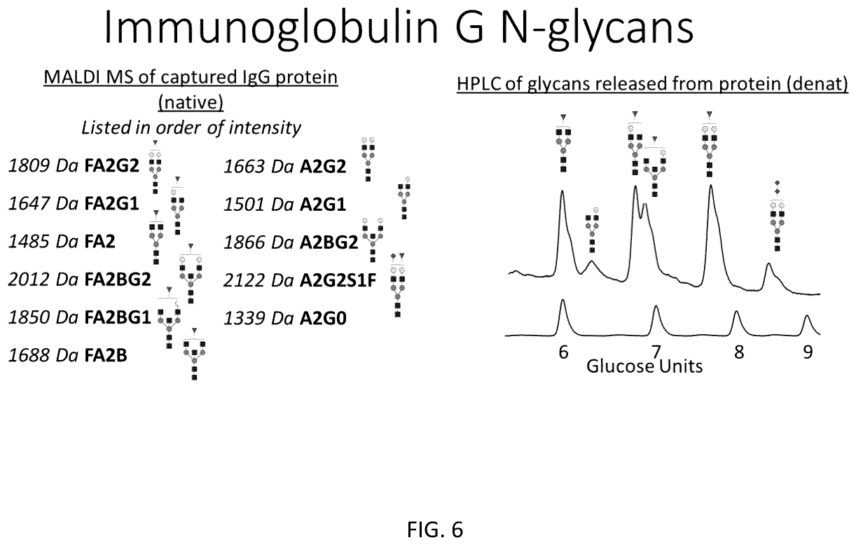

The most abundant types of N-glycans detected from the immunglobluin G captured from normal serum are shown in FIG. 6. These glycans were expected to be identified (primarily bisecting and bi-antennary) and are consistent with the glycan panels assessed for chronic disease. For comparison, the most abundant N-glycans detected by HPLC from purified (denatured) IgG that was PNGaseF digested and 2-AA labeled is shown in the right panel of FIG. 6. This gel denatured workflow required 2 days of preparation time. Overall, the approach of slide capture of IgG by antibody array methods yielded data consistent with to what has been reported, with minimal sample (1 μL) used, low cost antibody and rapid prep and assay times (as short as 6 hours). More inflammatory N-glycans (e.g., the G0 group) and sialylated N-glycans known to modulate IgG function can be readily detected.

The ability to perform N-linked glycan analysis of <100 ng of immuno-captured glycoprotein with equivalent glycan analysis as compared with in solution evaluation (as determined on a peak by peak basis by paired students T-test and Bland-Altman test). In addition, the signal to noise ratio is at least 10×.

In the present study, the system is optimized and the correct level of spotted antibody and MS conditions are determined to allow for the greatest level of sensitivity, specificity, and concordance with direct glycan analysis. Experimental variables in this study include: the amount of antibody spotted; the amount of antigen added to the wash conditions; the PNGAse F concentrations; and the incubation conditions (time). The first two proteins of interest include human IgG (hIgG) and A1AT. hIgG has a single site of glycosylation that is very well characterized. A1AT has three sites of glycosylation and similarly has been characterized. Antibody to either A1AT or hIgG are spotted onto glass slides as shown in FIG. 5B and FIG. 5C at concentrations from 300 ng down to 0.1 ng. In prior antibody array work, parameters included spot sizes of ~80-100 μm with a deposit volume of 350 pL and a final concentration of ~70 ng of antibody per spot. After PNGAse F treatment to remove the inherent glycosylation of the capture antibodies, the antibody spots (either for A1AT or hIgG) are covered with their respective proteins or another protein to control for specificity (IgG on A1AT antibody and vice versa). In all cases, fluorescently labeled (IRE-800 dye) protein are used, which allow for a quick determination if protein has bound to antibody by imaging on a scanner. In addition, although the level of antigen added to the slide will vary, in all cases a saturating amount of protein will eventually be used. The logic here is to look at the glycan from saturation amounts of antibody-captured protein. Slides are washed using several methods. Initial experiments use just 1×PBS but PBS is tested with up to 0.5% tween-20 (final wash is always water). The exact washing conditions are determined empirically and the best condition selected based upon greatest degree of binding and specificity of binding (e.g., no A1AT binding to the anti-IgG area and vice versa). This is detected using a Li-Cor Odyssey CLx reader, as the proteins are IRE-800 dye labeled.

Subsequently, slides are sprayed with PNGase F (0.1 μg/μL) using the HTX TM Sprayer and incubated for a time range of 1 hr to overnight under high humidity at 37° C. The optimal time is the incubation period that gives the lowest level of spot to spot variability while maintaining glycan profiles that are identical to that observed with free protein.

The MALDI matrix CHCA (7 mg/mL in 50% ACN/0.1% TFA is sprayed onto the slide using the TM sprayer. Subsequently, glycans are detected using a MALDI FTICR (7T solariX, Bruker Daltonics) (Broadband mode m/z 495-5000; positive ion mode; spatial resolution 20 μm; number of laser shots per spot: 20; data is viewed with fleximaging software version 4.1). In all cases samples are analyzed in replicates of 20 (at a minimum) and mean values used for comparison. In solution glycan analysis is analyzed both by direct analysis in the MALDI FT-ICR and by normal phase HPLC (after labeling of glycan with a fluorescent dye). Similarly, this is done in replicates of 20 to match the slide based analysis. Initial work involves protein diluted in PBS but will also utilize animal serum to test for any potential serum effect and also to optimize the wash conditions for serum use.

To determine the conditions required for accurate glycan information from antibody-captured antigen, the results are compared to the method of in-solution digestion and direct MS analysis, which is considered the “gold standard” for this purpose. To that end, for each protein, A1AT or hIgG, the top 10 glycans are given a percentile so that each glycan is given a percentage of the total glycans. For example, in the HPLC analysis in FIG. 5A, the di-sialylated biantennary glycan represents 45% of the total glycan pool, while the mono-sialylated bi-antennary glycan represents 31% and so on (the total of the 10 peaks is 100%). This percentile of glycan is done for the glycans analyzed by HPLC, direct MS, and by the present study. In all cases, as analysis is done in replicates of 20 (at a minimum), descriptive statistics are used on a peak-by-peak basis (for all 10 peaks) to compare methods. Initially, data distribution, bias, and variation are examined via a Bland and Altman plot (BA-A plot). Subsequently, the mean value of each glycan peak are compared from each method using a paired students T-test (using Graph-pad Prism 7.0). Additionally, replicates of each peak from each method are used to determine the coefficient of variation (CV) for each method. A CV of 20% is considered acceptable. An examination of all peaks is done after log transformation of all values (to help normalize the data as some peaks are much more abundant than others) and an analysis of all peaks from the direct MS analysis and the present method compared using correlation of coefficients analysis (Pearson correlation coefficient) and using the B-A plot. In all cases a p<0.05 will be considered significant.

Confirmation of capture is achieved by labeling both hIgG and A1AT with the IRE-800 dye. In this way, binding of antigen to antibody can be shown using the Li-Cor Odyessey CLx plate reader.

Sialic acids: One major issue with conventional MALDI-TOF based methods is the loss of sialic acid during ionization. This loss is minimal in the MALDI-FTICR due to the source configuration and cooling gases, but structures with more than two sialic acids are difficult to detect. To address this issue for the present method, a recently developed method is utilized, consisting of a linkage-specific in-situ ethylation derivatization of sialic acids for N-glycan mass spectrometry imaging of FFPE tissues. This method allows for the successful stabilization of sialic acids in a linkage specific manner, thereby not only increasing the detection range, but also adding biological relevance. This is a mild chemical reaction that can be easily done in solution or on-slide for glycoprotein preparations.

Spot to spot diffusion: One issue of concern is spot density and diffusion of glycan information from one spot to the next. This is tested by the use of side-by side capture of A1AT or hIgG. The logic of using these two proteins is that they have very distinct glycan profiles. Human IgG has ~30% core fucosylated biantennary glycan lacking galactose residues, as well as a significant portion with just a single galactose residue. These structures are not found on A1AT and therefore can be used as an indicator that no spot cross contamination has occurred. Use of the TM sprayer to apply PNGase F is expected to aid in decreasing diffusion issues.

Evidence that efficient de-glycosylation of antibodies have occurred: To ensure that efficient de-glycosylation of antibody and thus no signal from the capture antibody is seen, a capture slide is utilized where no sample is added to act as a control for the specificity of glycan detection.

Evidence of antibody specificity: To examine antibody specificity, in lieu of spraying with PNGase F to remove glycan, trypsin is sprayed to allow for spot by spot protein analysis. This is used to help confirm the specificity of antibody binding as well. Peptide profiles are evaluated by MALDI-FTICR, or off-slide by LC-MS (Orbitrap Elite).

Correlation with the off-slide analysis: Correlation is established between the on-slide and off-slide protein. This is optimized by varying the amounts and incubation time of the sprayed PNGase F, as well as antibody spot size and antibody concentration.

Variation in the actual spotting: Spotting of antibody is examined in sextuplet using IRE-800 dye labeled antibodies, enabling directly examination on the Li-Cor Odyssey CLx plate reader to determine how evenly the antibodies are spotted.

Multiple N-glycan isomers are possible, especially for branched and multifucosylated species. Initial work utilizes exoglycosidase to help resolve isomers (e.g. core versus outer arm fucosylation, etc). Exoglycosidases can be used on both free glycan in solution and on glycoproteins in protein microarrays. Use of ion mobility MS methods are an additional option.

Mass spectrometry platform: The MALDI-FTICR instrument provides maximum sensitivity. Once conditions are optimized, a MALDI-TOF (AutofleX III; a common MALDI-TOF instruments) capable of linear and/or reflector measurements including the rapifleX MALDI-TOF (a new platform with 5 micron and lower laser spot size capabilities) can be used in the assay.

The sensitivity of the glycan analysis is directly related to the level of antigen captured with the arrayed antibodies. In preliminary results, glycan was detectable from 1 ng of antibody. Spot sizes can be further balanced to ensure that at least 1-10 ng of protein can be captured.

### Example 2: Mass Spectrometry Analysis of Antibody Captured Serum Proteins

The following study examines 32 proteins that are of particular interest since their glycosylation is altered in hepatocellular carcinoma (HCC). An 8×4 array enables the creation of a single slide with four quadrants that can be treated in different ways. A first quadrant is left unsprayed with PNGase F, while a second quadrant is sprayed with trypsin instead of PNGase F to allow for confirmation of protein capture. A third quadrant is incubated without serum to control for efficient de-glycosylation of spotted antibodies. A fourth quadrant is used as a full experimental set. Proteins are captured from a complex mixture (human serum) and glycan data is generated from the individual captured proteins. N-linked glycan analysis can be obtained from 20 runs of healthy serum with less than 20% variation in peak quantification from multiple runs (on a peak by peak basis).

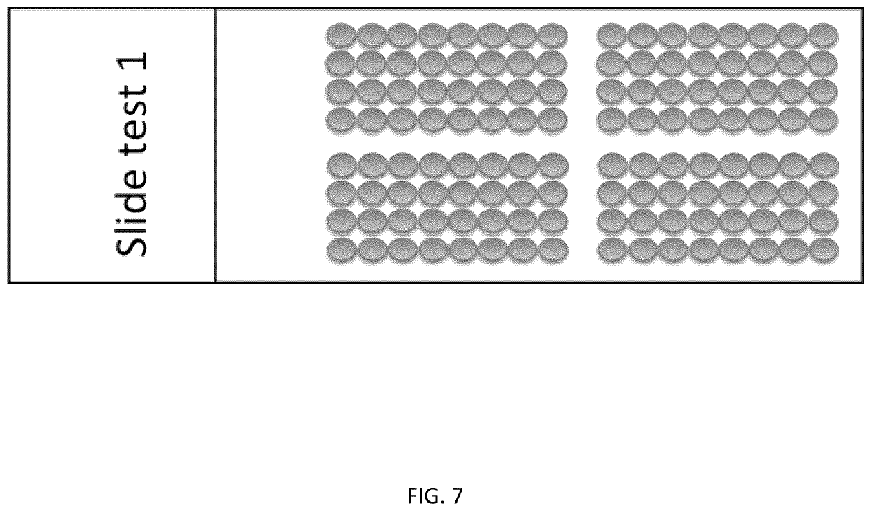

Experimental design: Antibodies are coated onto microscope slides (PATH, Grace Bio-Laboratories, Bend, OR) using a robotic arrayer (2470, Aushon Biosystems, Billerica, MA). Each slide contains 128 spots arranged in an 8×4 grid with 2.25 mm spacing between arrays. After printing, hydrophobic borders are imprinted onto the slides (SlideIm-printer, The Gel Company, San Francisco, CA) to segregate the arrays and to allow for multiple separate sample incubations on each slide. As shown in FIG. 7, the first three quadrants can be used for triplicate analysis while the last array is used for either a non PNGase F control or a trypsin control for protein identification on other slides. Multiple slides are used for analysis and the glycans compared across slides.

Statistical considerations: The goal of this study is to ensure the reproducible analysis of captured proteins. 20 repeated protein sample analysis produces replicate data from spot to spot and from slide to slide. Analysis of the top 10 glycans is used to percentile each glycan and the value used to determine reproducibility and CVs. A 20% CV is considered acceptable for this analysis.

Glycoproteins: The list of antibodies arrayed on the slide include A1AT, fetuin-A, hemopexin, Apo-J, LMW Kininogen, HMW Kininogen, apo-H, transferrin, IgG, IgM, IgA, fibronectin, laminin, ceruloplasmin, fibulin, angiotensinogen, Fibrillin-1, TIMP1, thrombospondin 1, galectin-3 binding protein, complement C1 R, clusterin, galectin 1, alpha-2-macroglobulin, Vitamin D binding protein, histidine rich glycoprotein, histidine rich glycoprotein, CD109, CEA, Cathepsin, AFP, and GP73.

Serum: Commercially purchased normal human serum (Sigma, Chemicals) is used in addition to a set of archived sera from patients with HCC (n=9), where glycan analysis on purified A1AT was previously performed. The controls sera is used in 20 replicates to establish assay reproducibility.

Mass spectrometry: The overall method and workflow is as shown in FIG. 1. The only difference is that instead of spotting protein onto specific locations on the slide, the entire slide is incubated with diluted serum. Briefly, human serum is diluted 2-fold into a buffer (1×PBS with 0.1% Tween-20, 0.1% Brij-35, species-specific blocking antibodies, and protease inhibitor) and incubated on an antibody array overnight at 4° C. Subsequently, the slides are washed 3× in 1×PBS and processed as in FIG. 1. As before, array sections are “untreated” with PNGase F to show glycan specificity.

Validation of glycan analysis: In this larger array, the glycan data is validated through the in-solution glycan analysis of purified protein. Most of the proteins can be purchased through commercial vendors, enabling in-solution analysis to provide validation of the slide-based analysis. The results are also compared via the MALDI-FTICR MS with that observed via lectin analysis using a lectin microarray system.

Data analysis: MS spectra are generated for each antibody captured protein. Data is imported into the SCiLs software package to determine glycan distribution for each replicate. From this, the coefficient of variation (CV) is determined for each peak.

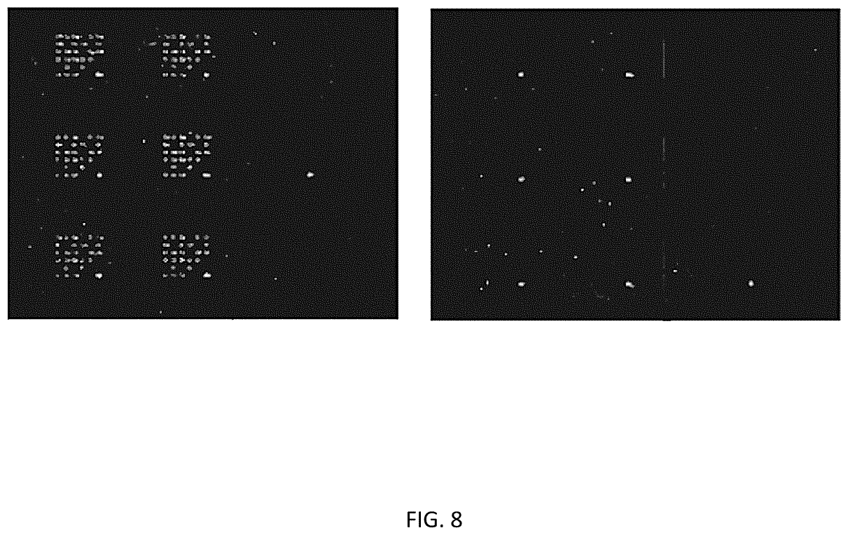

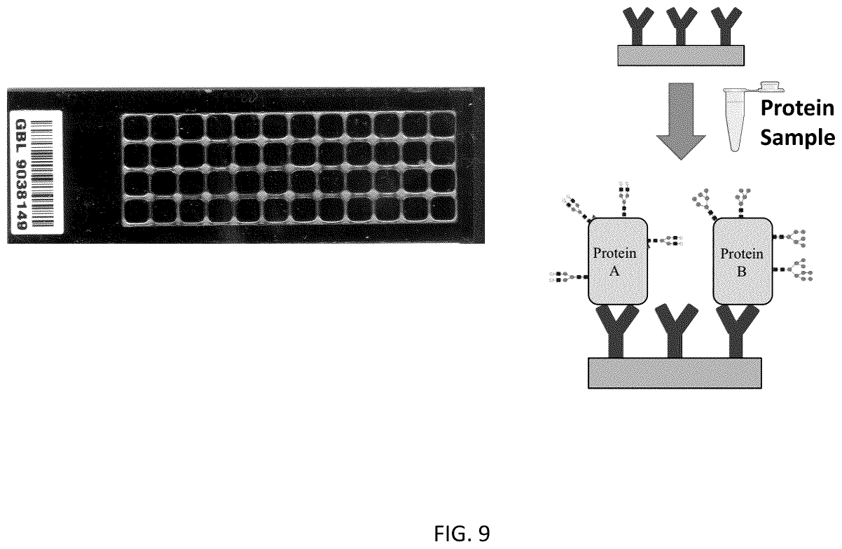

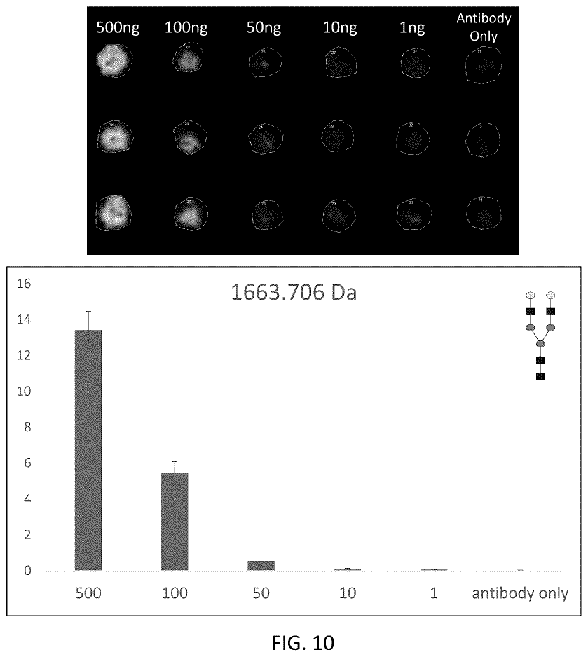

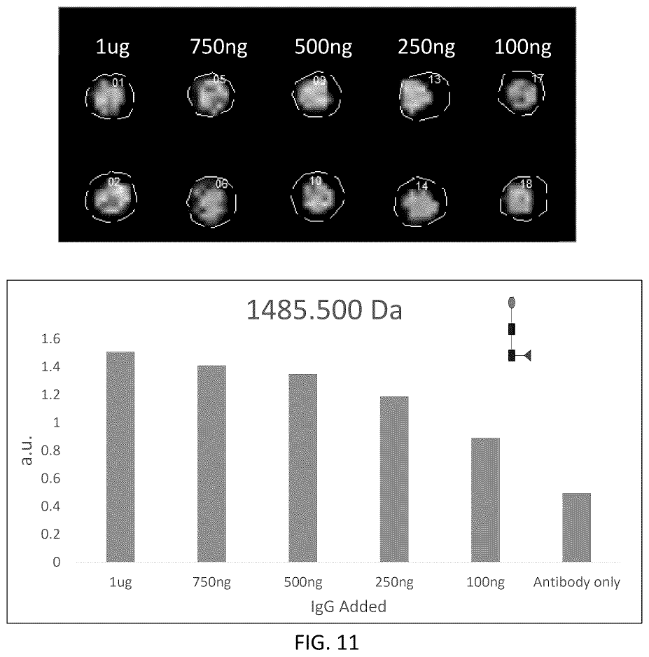

Adjustments are made to avoid issues include the detection of sialic acid containing glycan, bleeding of signal from spot to spot, and evidence for antibody specificity. In the case of sialic acid containing proteins, this is handled by linkage specific in-situ ethylation derivatization of sialic acids. The issue of spot to spot contamination is monitored by the placing of spots in locations that allow for comparison of known glycans (such as IgG and A1AT). In addition, duplicate arrays (other quadrant) are sprayed with trypsin to allow for protein capture information on a spot by spot basis. Background may be increased with less stringent wash conditions used. For observed high levels of non specific binding, more stringent wash conditions are used in the form of PBS with 0.001 to 0.1% tween-20 followed by a final rinse in mill-Q water. As a control, arrays are tested without serum added to control for glycan signals from capture antibody (similar to what is shown in FIG. 8).

The present study verifies on a small scale that glycan analysis can be performed on multiple proteins captured via antibodies on glass slides. The ability to do this has a tremendous impact upon biomarker discovery and validation, as many of the glycan changes observed in cancer cannot be easily detected by lectins. In addition, the simple and rapid nature of this assay has tremendous translation potential and has the potential to become an independent biomarker platform.

### Example 3: Development of N-glycan Analysis as Applied to Immune Cell Subtypes

Glycan analysis of white blood cells has remained limited to primarily individual cell lines, for example THP-1 monocytes, and there are no methods reported that would allow glycan profiling of immune cells analogous to IgG profiling. The following study provides a method of cell N-glycan profiling, which is termed Glyco-Cell Typer, and involves the capture of specific cell types on slides using directed antibody capture followed by glycan release and analysis using an established workflow. The present study utilizes well defined B, T and macrophage cell lines. Total white cell isolates from blood via Ficoll collection are also evaluated.

Antibody to specific cell targets are applied to slides at varying levels initially (100-500 ng) used to capture specific cell types. Anti-CD4 (MABF417, Anti-CD4 Antibody (human), PE, clone OKT4) are used to capture CD4+ T-cells, anti-CD8(MABF1676, Anti-CD8 (human), PE, clone SK1 Antibody) are used to capture CD8+ T-cells, anti-CD19 (CBL582, Anti-CD19 Antibody, clone HD37) are used to capture B cells, and anti-CD14 are used to capture monocytes (MAB1219, Anti-CD14 Antibody, clone 2D-15C).

Initially work is performed with cultured cell lines. CD4+ T-cells are Sup-T1 cells (ATCC #SUP-T1 [VB] (ATCC® CRL-1942). CD8 cells are TALL-104 (cells ATCC® CRL-11386). B-cells are the C1R-neo cells (ATCC® CRL-2369™). Monocytes are the THP-1 cell lines (ATCC® TIB-202™). Total white blood cells (i.e, PBMC's) are isolated from plasma using a Ficoll-Paque collection tube and differential centrifugation.

The antibodies are attached to the slide using the workflows described elsewhere herein. After incubation with a cell population, slides are rinsed in PBS initially. The next step is fixation with neutral buffered formalin, followed by rinsing in Carnoy's solution, which is both a fixative and delipidating solution and does not disrupt cell morphology. Total PBMCs from the Ficoll layer are smeared and dried onto slides directly, analogous to cell culture slides. The effectiveness of the antibody enrichment of the cultured cells is evaluated by comparing the N-glycan signature of cell smears (fixed and washed as above) with more traditional glycomic analysis by HPLC. For most of the evaluated antibodies, Ficoll fractions of PBMCs can be used to simultaneously glyco-type the constituent immune cell populations.

The study determines the minimal amount of antibody required for capture of cells and for glycan detection. Analysis is done in triplicate, and the coefficient of variation (CV) is calculated for each peak (from each glycoprotein). A CV of 10% is considered acceptable.

The study also compares the array findings to results from solution-phase analysis. Glycan analysis of the cell types are performed “off slide” and examined by MALDI FT-ICR in the same way they are examined on slide. The results are expected to demonstrate 100% concordance with glycan presence with a 15%<CV between replicate spots (using mean values). That is, if a glycan is observed in the solution based analysis, it should be detected “on slide.” In all cases, the solution based analysis acts as the gold standard for comparison. A 15%<CV is considered acceptable as this is the range normally observed for the solution based analysis.

The sensitivity of the glycan analysis is directly related to the number of cells captured with the arrayed antibodies. In preliminary results, glycan from glycoprotein standards could be detected from 1 ng of antibody. Cell numbers are titered to determine a LOI for each cultured cell type and are evaluated with the amount of antibody that is needed to be spotted to achieve this. Maintaining cell integrity during capture and rinsing prior to PNGaseF treatment is achieved by fixation in formalin after antibody binding.

### Example 4: Direct N-glycan Analysis of Cell Lines on Slides

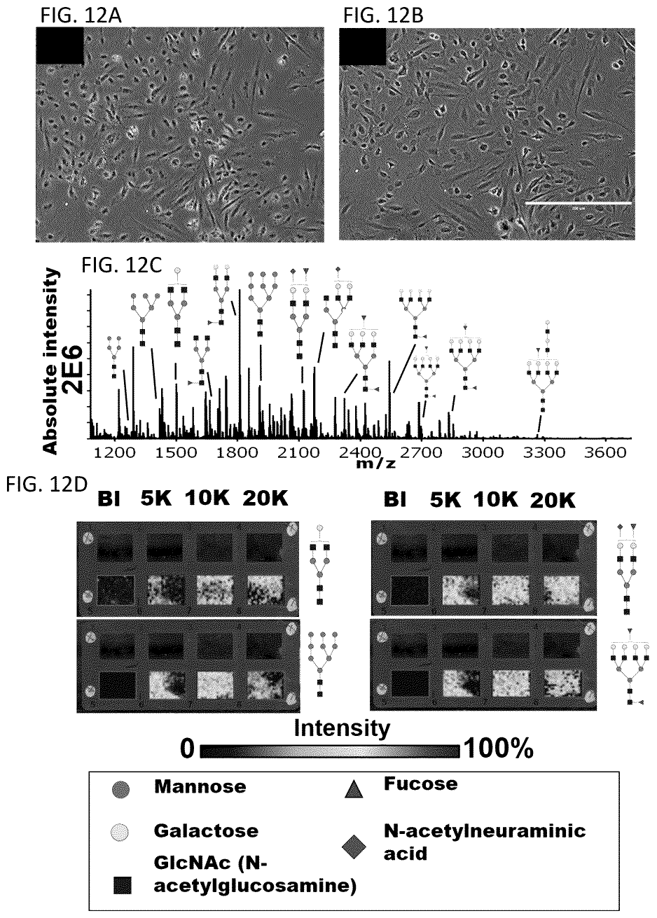

N-glycan profiles are obtainable from simplified sample preparation of cells in culture. Primary aortic endothelial cells (ATCC) were grown on a slide with eight chambers for cell growth. Cells were plated at densities of 5k, 10k, and 20k per mL. Cells were allowed to proliferate for 7 days. Cells were fixed in neutral buffered formalin, imaged by microscopy, and delipidated using Carnoy's solution, a fixative that also delipidates (FIG. 12A through FIG. 12D). Data shows the cells remain in place on the slide without disruption to morphology. Cells were then coated with a thin molecular layer of PNGase F (HTX Technologies), digested two hours, and coated with a thin coat of MALDImatrix. Cells were analyzed by MALDI coupled to a Fourier Transform Ion Cyclotron Resonance (FT-ICR) mass spectrometer in positive ion broadband mode over mass-to-charge range 499-5,000 with a transient of 1.20 seconds. FIG. 12C shows that complex N-glycan profiles are obtainable from a single layer of cells. Most N-glycans do not appear in the media blank (FIG. 12D). Higher spatial resolution time-of-flight instruments allow targeted imaging of a single cell from culture.

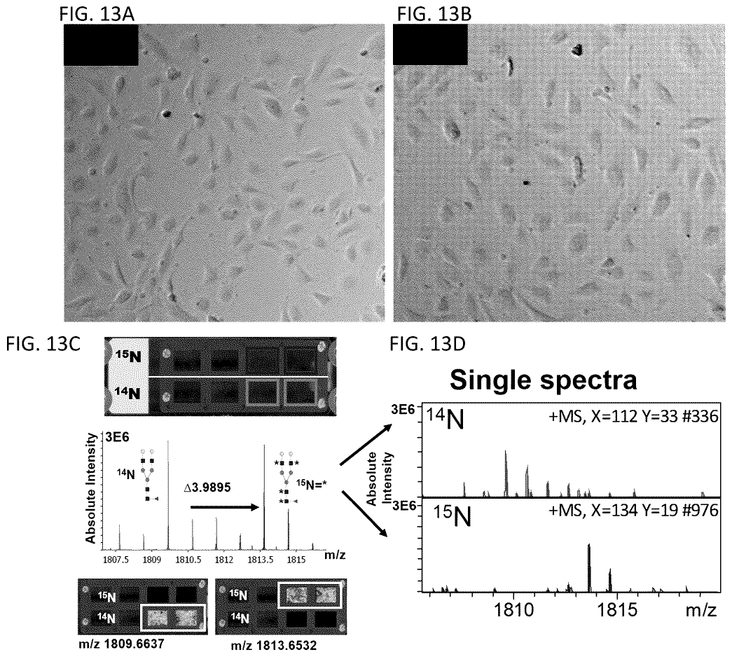

Stable isotopic labeling was examined in cell culture detectable from single cell layers by MALDI IMS (FIG. 13A through FIG. 13D). Endothelial cells were prepared and plated at 10k per mL. However, N15 labeled glutamine (amide side chain) was used to incorporate a stable isotope to all GlcNac, sialic acids, and GalNac. Cells showed no difference in proliferation after 5 days (FIG. 13A). Native and stable isotopes for m/z 1809 G2F showed detection of complete incorporation. Examination of single spectra suggested that intensities at the single spectra level were similar to overall intensities. This demonstrates that quantitative changes in N-glycosylation can be detected using a combination of simplified workflows detected by MALDI IMS.

### Example 5: Analysis of Cultured Cells on Slides for Direct Glycan Measurements

The following study analyzes cultured or captured HEk293, CHO, and human aortic endothelial cells and produces a glycan profile from a minimal amount of cells cultured or captured on a solid substrate. This workflow eliminates lengthy work needed to produce a glycan profile and significantly reduces the number of cells needed. In all cases, the total number of cells is compared to the signal from all types of N-glycans that permit the quick determination of the glycan profile of the cell type; based on preliminary data, detection is expected for a minimum of 50 N-glycans. For cell culture, cells seeded onto plate are varied from 1,000 to 20,000 cells/mL. For captured cells, routine histological techniques are investigated, such as cell slurries, swabbing, smearing, and Cytospin to apply cells to solid substrates.

For cultured or captured cells, detectability is determined with coatings compatible with cell culture and attachment of cells to microscope slide areas. Glass and indium tin oxide coated slides (used for MALDI TOF) are evaluated for broad application in all laboratories. Removable adhesive reticules are placed on the slide (e.g., FlexWell, Electron Microscopy Sciences), followed by coating for cell culture such as gelatin, collagen, or chemicals that facilitate cell attachment (poly-l-lysine, poly-ornithine). Selective removal of analytes that limit detection of N-glycans is accomplished using washing techniques, such as using Carnoy's solution, which is both a fixative and delipidating solution and does not disrupt cell morphology. Other solutions that do not disrupt cell morphology include neutral buffered formalin, paraformaldehyde and cytology fixatives based on ethanol and polyethylene glycol.

The study also investigates the effect of PNGase F sprayed onto cell layers, digestion times, and matrix coating. Cells are examined before and after each step to ensure cells retain morphology. This also facilitates downstream high spatial resolution imaging of cells. Signal is compared to that obtained from profiling a cell pellet through standard protocols. Typically, detection of the glycan profile takes less than an hour for a slide area of 65×25 mm. For cell culture work, testing is done on a minimum of six replicates that are independently grown in separate culture dishes. For cells captured by histology methods, replicates are examined in sextuplet from the same source for comparison.

Adaption of approaches for simplified quantification of target glycans are investigated. Stable isotopic labeling and label free approaches are tested for cell culture detectable by IMS; this facilitates detailed studies on glycan profiles affected by genetic manipulation or altered pathways of synthesis. Primary cells are grown using glutamine labeled with ¹⁵N at the amide site (Cambridge Isotopes). This incorporates the ¹⁵N label into GlcNAc residues, sialic acids, and N-acetylgalactosamine (GalNAc). This approach tags all core GlcNAc involved in new N-glycan turnover, new modified GlcNAc and SIA capping, and new GalNAc extensions (mostly O-linked), resulting in a 0.9970 Da shift per residue change. ¹⁵N labeling is done with cell culture conditions for downstream detection by IMS. FIG. 13A through FIG. 13D demonstrate IMS detection of the ¹⁵N label into human primary endothelial cell culture.

Label-free quantitative methods are tested by spiked heavy-isotopically labeled glycans as a single target glycan or as a mix of heavy isotopically labeled glycans added to the matrix spray and sprayed onto cells captured on slides as an internal standard. This allows quantification relative to a standard(s) across cell conditions. Second, mixtures of common glycans are spotted as a calibration curve. This approach compares standard glycans spotted onto cells (without glycan release) versus spotted as an independent external calibration curve. Spotting onto cells and comparing with signal off cells permits evaluation for ion suppression effects due to the sample matrix. Sensitivity of label free methods for cell based work is determined using cell targeting by IMS and modulating laser size to include specific numbers of cells, and incrementally decreasing cell numbers to determine limits of detection, limits of quantification, and reproducibility. Glycan amounts are extrapolated relative to total protein content and/or cell numbers.

The present study demonstrates the minimum amount of cells yielding equivalent profiles obtained on-tissue. In preliminary data, complex signals were detected from 5,000 cells that were necessary to cultivate for a week to 60% confluency for biological work. This provides signal counts from the FT-ICR of 3.3E6. For evaluation of glycan profiles during cell density experiments, and cell capture experiments, a CV of ≤15% is considered acceptable, matching current on-tissue and in solution results. Limits of detection are computed as cell density on solid substrate.

### Example 6: A Novel Platform for Multiplexed N-glycoprotein Biomarker Discovery from Patient Biofluids by Mass Spectrometry Imaging of Antibody Arrays

A new platform for N-glycoprotein analysis from serum using matrix-assisted laser desorption/ionization mass spectrometry imaging (MALDI MSI) workflows and antibody arrays is described. Serum N-glycoproteins can be specifically immunocaptured by antibodies on glass slides to allow N-glycan analysis in a protein-specific and multiplexed manner. Development of this technique has focused on characterizing two abundant and well-studied human serum glycoproteins, alpha-1-antitrypsin and immunoglobulin G. Using purified standard solutions and one microliter of human serum, both glycoproteins can be immunocaptured and followed by release of N-glycans by PNGase F. N-glycans are detected on a MALDI FT-ICR mass spectrometer in a concentration-dependent manner while maintaining specificity of capture. Importantly, the N-glycans detected via slide-based antibody capture was identical to that determined by direct analysis of the spotted standards. As a proof of concept, the workflow was applied to serum samples from individuals with liver cirrhosis to accurately detect a characteristic increase in IgG N-glycans. This novel approach to protein-specific N-glycan analysis from an antibody array can be further expanded to include any glycoprotein for which a validated antibody exists. Additionally, this platform can be adapted for analysis of any biofluid or biological sample that can be analyzed by antibody arrays.

Glycosylation is one of the most common post-translational modifications and often consists of the covalent addition of an oligosaccharide (glycan) to either an asparagine (N-linked) or serine/threonine (O-linked) residue. N-linked glycans have been well-established to change with the progression of cancer and other diseases (Kailemia M J et al., Analytical and bioanalytical chemistry, 2017, 409(2), 395-410; Adamczyk B et al., Biochimica et Biophysica Acta (BBA)-General Subjects, 2012, 1820(9), 1347-1353; Kuzmanov U et al., BMC medicine, 2013, 11(1), 31; Ohtsubo K et al., Cell, 2006, 126(5), 855-867), and studies indicate that the N-glycan component of a glycoprotein may act as a specific disease biomarker more than the protein alone (Adamczyk B et al., Biochimica et Biophysica Acta (BBA)-General Subjects, 2012, 1820(9), 1347-1353; Meany D L et al., Clinical proteomics, 2011, 8(1), 7). This has been shown in the success of fucosylated alpha-fetoprotein (AFP) as a biomarker for liver cancer (Taniguchi N, Proteomics, 2008, 8(16), 3205-3208; Aoyagi Y et al., Cancer: Interdisciplinary International Journal of the American Cancer Society, 1998, 83(10), 2076-2082), yet most N-glycans present on protein biomarkers remain unexplored. Current techniques for analysis of N-glycans and their carrier proteins are often time-consuming or require large amounts of sample (Kailemia M J et al., Analytical and bioanalytical chemistry, 2017, 409(2), 395-410; Kuzmanov U et al., BMC medicine, 2013, 11(1), 31; Mariño K et al., Nature chemical biology, 2010, 6(10), 713; Geyer H et al., Biochimica et Biophysica Acta (BBA)-Proteins and Proteomics, 2006, 1764(12), 1853-1869), which limits the ability to analyze significant numbers of patient samples for the discovery of novel disease biomarkers. High throughput methods have also utilized differential lectin binding to identify carbohydrate structural motifs (Reatini B S et al., Analytical chemistry, 2016, 88(23), 11584-11592; Chen S et al., Nature methods, 2007, 4(5), 437; Hirabayashi J et al., Journal of biochemistry, 2008, 144(2), 139-147). These approaches are limited to the variable and low binding affinities of most lectins, and they cannot be used to report true structural composition or glycan carrier (i.e. N-glycan, O-glycan, or glycosphingolipid) information (Reatini B S et al., Analytical chemistry, 2016, 88(23), 11584-11592; Chen S et al., Nature methods, 2007, 4(5), 437; Hirabayashi J et al., Journal of biochemistry, 2008, 144(2), 139-147).

The technology of matrix-assisted laser desorption/ionization (MALDI) mass spectrometry imaging (MSI) has emerged in recent decades to become a powerful technique for analyte detection and localization across tissue sections (Caprioli R M et al., Analytical chemistry, 1997 69(23), 4751-4760; Seeley E H et al., Journal of Biological Chemistry, 2011, 286(29), 25459-25466; Buchberger A R et al., Analytical chemistry, 2017, 90(1), 240-265; Balluff B et al., Histochemistry and cell biology, 2011, 136(3), 227; Walch A et al., Histochemistry and cell biology, 2008, 130(3), 421). This technique creates two-dimensional heat maps of an analyte's intensity across a tissue sample. A previously developed method for the spatial analysis of N-glycans in tissue sections has been implemented and adapted by many labs across the world. (Powers T W et al., PloS one, 2014, 9(9), e106255; Drake R R et al., In Advances in cancer research, 2017, (Vol. 134, pp. 85-116). Academic Press; Powers T W et al., Analytical chemistry, 2013, 85(20), 9799-9806; Powers T et al., Biomolecules, 2015, 5(4), 2554-2572; Heijs B et al., Analytical chemistry, 2016, 88(15), 7745-7753; Gustafsson O J et al., Analytical and bioanalytical chemistry, 2015, 407(8), 2127-2139) As is common to any method relying on enzymatic release of N-glycans, linking N-glycan signatures to their carrier proteins remains laborious and requires extensive additional analysis (Heijs B et al., Analytical chemistry, 2016, 88(15), 7745-7753; Angel P M et al., In Tissue Proteomics, 2017, (pp. 225-241). Humana Press, New York, NY). Leveraging that MALDI MSI can detect N-glycans from the solid surface of a tissue on a slide, it was hypothesized that N-glycan profiles could be detected from target glycoproteins captured on a slide-based antibody microarray. This would bridge the gap in linking N-glycan signatures to their proteins, as the location of the detected N-glycans along the array would indicate which immunocaptured glycoprotein they were released from. The following study reports a novel platform for glycoprotein biomarker discovery by coupling the localization of MALDI MSI with the protein capture specificity of an antibody array for use with patient samples.

The materials and methods are now described.

### Materials

Nitrocellulose-coated microscope slides (PATH microarray slides) and well slide modules (ProPlate Multi-Array Slide System, 24-well) were obtained from Grace Bio-Labs (Bend, OR). Trifluoroacetic acid, α-cyano-4-hydroxycinnamic acid, octyl-β-D-glucopyranoside, human alpha-1-antitrypsin, and stock human serum were obtained from Sigma Aldrich (St. Louis, MO). HPLC grade water, HPLC grade acetonitrile, bovine serum albumin (BSA), and phosphate buffered saline (PBS) were obtained from Fisher Scientific (Hampton, NH). ICG NHS ester for protein labelling was obtained from Li-cor Biosciences (Lincoln, NE) as a custom IRDye 800CW derivative. Peptide-N-glycosidase F (PNGaseF) Prime™ was cloned, expressed, and purified in-house as previously described (Powers T W et al., Analytical chemistry, 2013, 85(20), 9799-9806). Anti-human A1AT was obtained from Genway Biotech (San Diego, CA). Human immunoglobulin G was obtained from (Jackson ImmunoResearch (West Grove, PA) and anti-human IgG from Bethyl Laboratories (Montgomery, TX). Cirrhotic patient serum was obtained from Dr. Amit Singal (University of Texas Southwestern Medical Center, Dallas, TX). Serum samples were obtained via a study protocol approved by the UTSW Institutional Review Board, with written informed consent obtained from each subject. Diagnosis of liver cirrhosis was based on liver histology or clinical, laboratory, and imaging evidence of hepatic decompensation or portal hypertension. Each patient had a normal ultrasound; if serum AFP was elevated, a CT or MRI showed no liver mass. Further patient details regarding these samples can be found in Wang M et al., Journal of immunological methods, 2018, 462, 59-64.

### Microarray Preparation

Nitrocellulose-coated microscope slides were obtained and wells were created with a 24-well slide module clipped to the slide. Antibodies were manually spotted in wells at 200 ng per 1.5 μL spot. Spots were then left to adhere overnight at 4° C. in a humidity chamber made from a 12×9×3.5 cm western blot incubation box lined with a Wypall×60 paper towel and 2 rolled Kim Wipes saturated with distilled water. Slides were then air dried at room temperature and rinsed with 0.1% octyl-β-D-glucopyranoside in 1×PBS (hereinafter “PBS-OGS”) to remove any unbound protein from slide.

### Sample Preparation and Glycan Release

Slides were blocked with 1% BSA (prepared in PBS-OGS) for one hour with gentle shaking. Slides were then washed in baths of PBS (3 mins×2 baths) and double distilled water (1 min×1 bath) and let air dry. Once dry, samples were added to wells and incubated at room temperature for 2 hours in a humidity chamber with gentle shaking. All samples were diluted in PBS, with sample volumes of 100 μL added to wells. Slides were then washed in baths of PBS-OGS (1 min×1 bath), PBS (3 mins×2 baths), and double distilled water (1 min×1 bath) and let air dry. An additional water rinse was performed after the removal of the well module from the slide to remove any residual salt. In order to cleave N-glycans from captured proteins, PNGaseF Prime™ (0.1 μg/μL, prepared in HPLC grade water) was applied using an automated sprayer (M3 TM-Sprayer, HTX Technologies, Chapel Hill, NC) to retain localization with spraying parameters 15 passes at 45° C., 10 psi, flow rate 25 μL/min, and 1200 mm/min velocity. Slides were then incubated overnight at 37° C. in humidity chambers made in cell culture dishes with Wypall ×60 paper towels and 2 rolled Kim Wipes saturated with distilled water.

### Mass Spectrometry Sample Preparation and Imaging

MALDI matrix α-cyano-4-hydroxycinnamic acid (CHCA, 7 mg/mL in 50% acetonitrile/0.1% trifluoroacetic acid) was applied to slides using the same automated sprayer (M3 TM-Sprayer, HTX Technologies, Chapel Hill, NC). Application parameters were 2 passes at 77° C., 10 psi with a 1300 mm/min velocity and flow rate 100 μL/min. Slides were imaged on a solariX Legacy 7T FT-ICR (Bruker Daltonics) mass spectrometer equipped with a matrix-assisted laser desorption/ionization (MALDI) source. Sampling was done using a SmartBeam II laser operating at 2000 Hz with laser spot size of 25 μm. Images were collected using a smartwalk pattern at a 250 μM raster with 200 laser shots per pixel. Samples were analyzed in positive ion, broadband mode using a 512 kword time domain spanning m/z range 500-5000. An on-slide resolving power of 58,000 at m/z 1501 was calculated.

### Data Analysis

Images of N-glycan localization and intensity were visualized using FlexImaging v4.1 (Bruker Daltonics), with data imported into FlexImaging reduced to 0.98 ICR Reduction Noise Threshold. Images were normalized to total ion current and N-glycan peaks were selected manually based on their theoretical mass values. Data was then imported into SCILS Lab software 2017a (Bruker Daltonics) for quantification of peaks at individual spots. Each spot was designated a unique region and area under peak values for masses of interest were exported from each region into Microsoft Excel.

### HPLC Orthogonal Confirmation

HPLC analyses on released labelled N-glycans were performed using a Waters Alliance HPLC system as previously described (Comunale MA et al., PloS one, 2010, 5(8), e12419).

The Results Are Now Described.

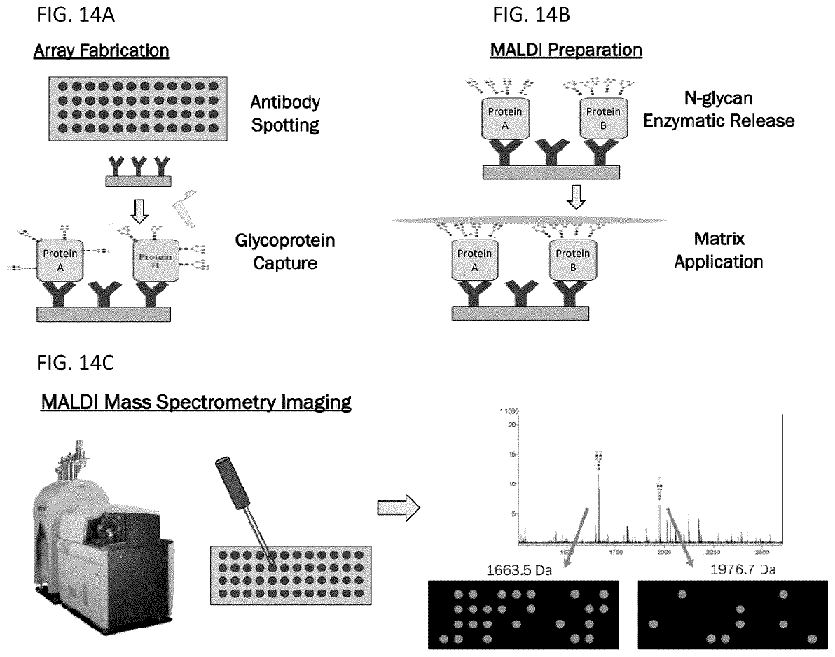

The novel workflow for specific glycoprotein capture and mass spectrometry imaging (MSI) is illustrated in FIG. 14A through FIG. 14C. The workflow is founded upon a similar MALDI MSI workflow for N-glycan imaging on tissue (Powers T W et al., Analytical chemistry, 2013, 85(20), 9799-9806), and consists of three major steps. The first (shown in FIG. 14A) involves antibody spotting and glycoprotein capture localized to their antibody spots. The second (FIG. 14B) consists of enzymatic release of N-glycans in a localized manner and matrix coating of the slide, trapping released glycans in area of their release. FIG. 14C shows the third step of MALDI MSI analysis of the slide, where an overall spectra is obtained with images correlating to each m/z peak. Images obtained from MALDI MSI depict the abundance of an N-glycan across a slide with color intensity, creating a heat map for each N-glycan detected. This allows for the visualization of N-glycans released from immunocaptured glycoproteins in an array type format, where N-glycans of interest can be linked back to their protein carriers.

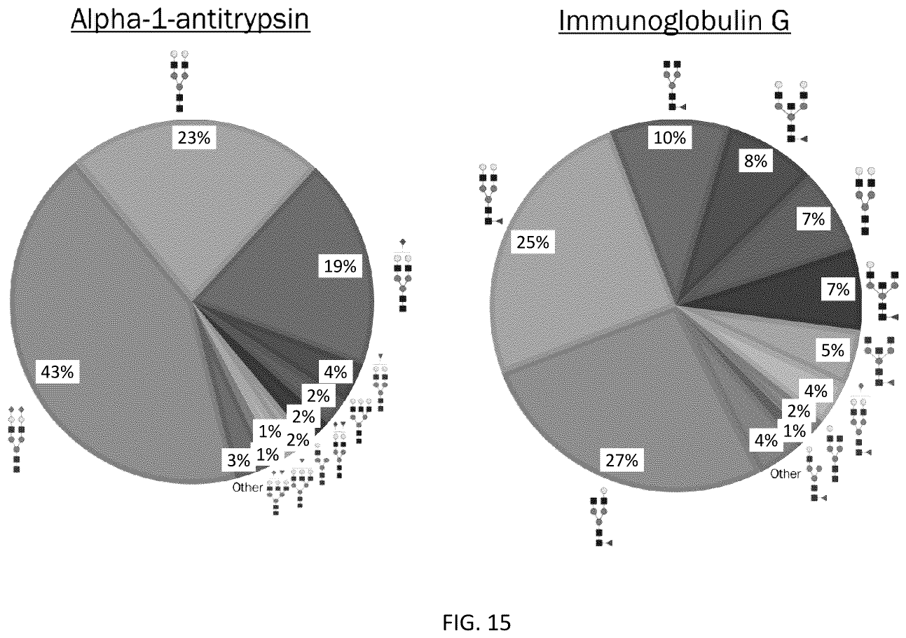

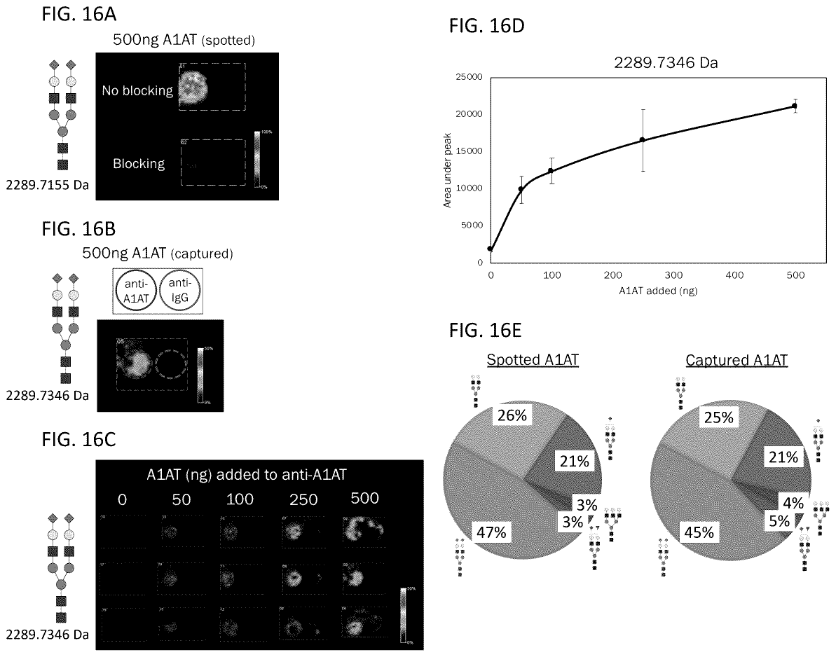

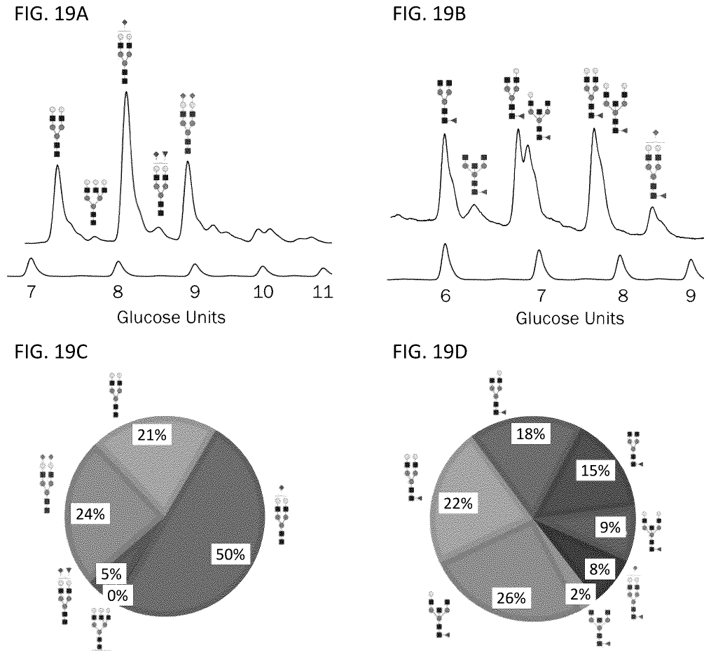

Initial experiments were done using human alpha-1-antitrypsin (A1AT) and immunoglobulin G (IgG) as they are abundant glycoproteins in human serum with well-characterized N-glycosylation sites (Comunale MA et al., PloS one, 2010, 5(8), e 12419; Clerc, F et al., Glycoconjugate journal, 2016, 33(3), 309-343; McCarthy C et al., Journal of proteome research, 2014, 13(7), 3131-3143; Mittermayr S et al., Journal of proteome research, 2011, 10(8), 3820-3829; Saldova R et al., Journal of proteome research, 2015, 14(10), 4402-4412). These glycoproteins have distinct N-glycan profiles from each other, as shown in FIG. 15 and FIG. 19A through FIG. 19D with orthogonal HPLC confirmation showing unique N-glycan profiles. The N-glycan profiles in FIG. 15 were obtained from MALDI MSI of spotted A1AT and IgG, showing all detected N-glycans comprising more than 1% of overall glycan signal. Proposed structures for these N-glycans are shown, with the core fucose linkage on IgG N-glycans assigned based on orthogonal HPLC characterizations of this glycoprotein (FIG. 19A through FIG. 19D) as well as other literature sources (Mittermayr S et al., Journal of proteome research, 2011, 10(8), 3820-3829; Saldova R et al., Journal of proteome research, 2015, 14(10), 4402-4412). Antibody capture of glycoproteins was performed similar to immunoassay procedures used in other array formats (Chen S et al., Nature methods, 2007, 4(5), 437; Wang J et al., PROTEOMICS-Clinical Applications, 2013, 7(5-6), 378-383). Antibodies were manually pipetted at 200 ng per spot, each spot a 1.5 μL volume. Wells were created with clip-on well modules and antibodies spotted within the wells. Slides were blocked with 1% BSA to prevent nonspecific binding to the slide or other antibodies. FIG. 16A illustrates that the slide is sufficiently blocked to prevent A1AT binding when the protein was spotted directly to the slide followed by a wash to remove unbound protein. When A1AT was added to a well as a 100 μL sample, capture specificity to its antibody was seen with A1AT N-glycans localized to anti-A1AT but not an adjacent anti-IgG spot, shown in FIG. 16B. Circles were added to indicate the position of spotted anti-A1AT (red) and anti-IgG (blue) within the well. FIG. 16C contains a dilution series of A1AT added to its antibody, illustrating the successful capture of a glycoprotein and N-glycan detection localized to capture spots. A main N-glycan signature was from m/z 2289.7346(Hex5HexNAc4NeuAc2+3Na), which is depicted in FIG. 16C. This glycan represents approximately 47% of the total glycan pool on A1AT and this peak can easily be observed at 50 ng of captured protein. This correlates to approximately 16 femtomoles of that glycan, which highlights the sensitivity of this platform. N-glycan signal intensities within each spot were quantified using the area under the peak. As shown in FIG. 16D, N-glycan signal from immunocaptured A1AT was detected in a concentration-dependent manner, with a signal plateau observed as the antibodies (spotted at 200 ng) became saturated. The profile of the most abundant N-glycans detected on this captured glycoprotein showed strong agreement with those seen on spotted glycoprotein (FIG. 16E). However, N-glycan of m/z 1809.6923(Hex5dHex1HexNAc4+Na) was excluded from this analysis as it is highly abundant on the capture antibody and thus would confound the comparison of spotted to captured profiles. An orthogonal analysis of the N-glycan profile of A1AT was performed on HPLC (FIG. 19A through FIG. 19D).

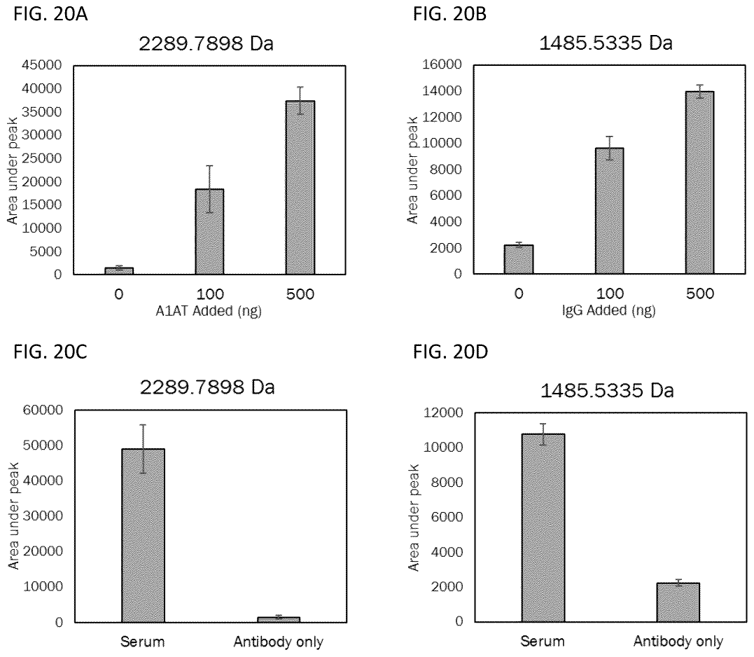

To illustrate the potential of this platform to become a multiplex array for the simultaneous analysis of multiple glycoprotein targets, side-by-side capture of two glycoproteins was tested. Human A1AT and IgG were used and antibodies to the two proteins were spotted side-by-side within each well as depicted in FIG. 17A. Mixtures containing both A1AT and IgG standards were added to wells in triplicate 100 μL volumes at the concentrations shown in FIG. 17B and FIG. 17C, with red and blue circles added to indicate antibody positions. N-glycans were detected from both glycoproteins localized to their individual capture spots, with an N-glycan signature unique to A1AT shown in FIG. 4B (m/z 2289.7898, Hex5HexNAc4NeuAc2+3Na) and unique to IgG in 4C (m/z 1485.5335, Hex3dHex1HexNAc4+Na). Quantifications to compare the protein signal to antibody background signal for these images are shown in FIG. 20A through FIG. 20D. Specificity of capture was observed by the lack of protein-specific N-glycan signals on the opposite antibodies as well as the surrounding slide itself. As the goal of this platform is application to biological samples for biomarker discovery, stock human serum was also used for side-by-side capture of glycoproteins A1AT and IgG from a more complex mixture. Commercially-obtained human serum was diluted in PBS (1:100) and added to the wells containing both A1AT and IgG antibodies as previously shown. FIG. 17D and FIG. 17E depict N-glycan signatures associated with both glycoproteins captured from just 1 μL of serum, again showing great specificity of capture.

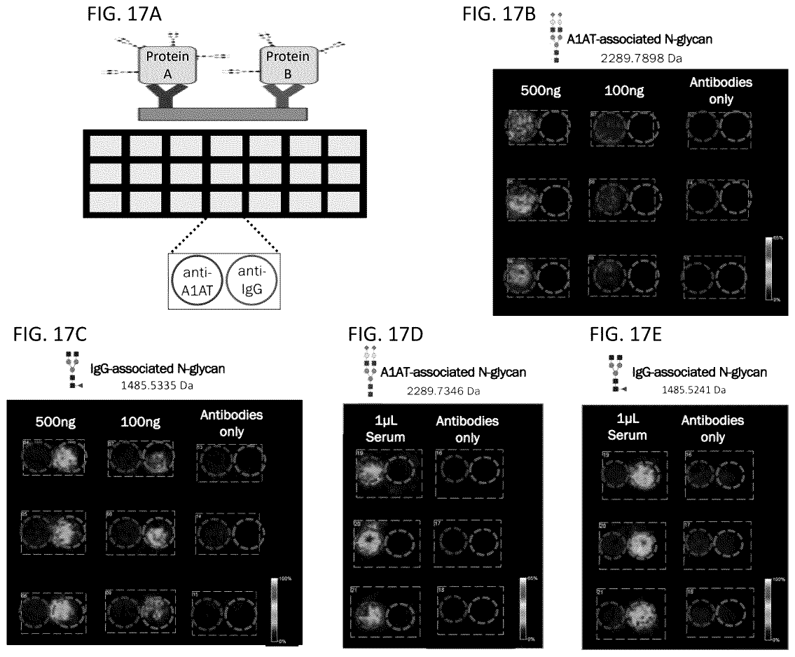

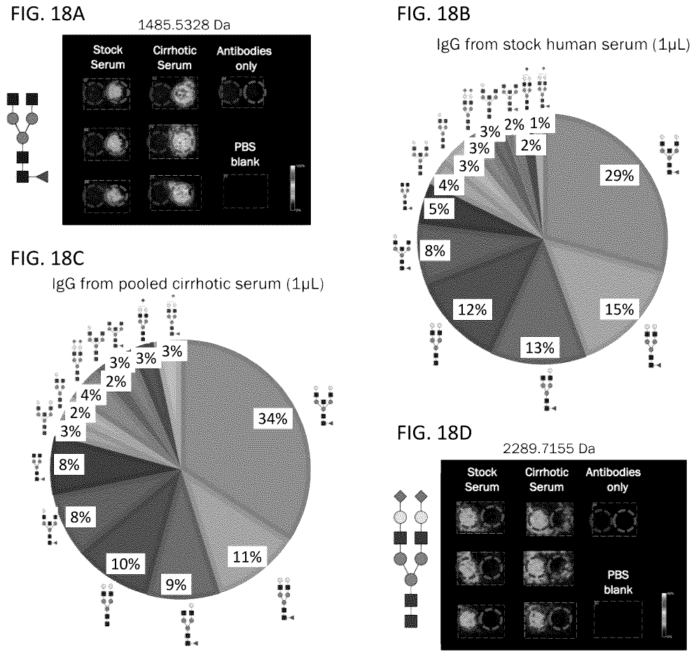

An application for this new platform is the discovery of disease-specific changes in N-glycans on proteins captured from patient biofluid samples. As a proof of concept, serum was pooled from 5 patients with liver cirrhosis, and 1 μL of the pooled sample was added to the array in the triplicate. Serum samples were diluted 1:100 in PBS prior to addition to the wells. Glycoproteins were again specifically captured by their antibodies with detectable levels of distinct N-glycans, with imaging data and overall N-glycan profiles of IgG shown in FIG. 18A through FIG. 18D. Notably, an increase in an IgG nongalactosylated N-glycan m/z 1485.5328 (Hex3dHex1HexNAc4+Na) was observed in the cirrhotic serum compared to stock human serum, FIG. 18A through FIG. 18C. This particular N-glycan has been previously described to increase in cirrhotic serum (Mehta A S et al., Journal of virology, 2008, 82(3), 1259-1270; Lamontagne A et al., PloS one, 2013, 8(6), e64992) and this new platform showed agreement with those findings. A subsequent decrease in galactosylated biantennary N-glycans (m/z 1809.6293Hex5dHex1HexNAc4+Na and 1647.5545Hex4dHex1HexNAc4+Na) was also observed (FIG. 18B and FIG. 18C). A1AT was also successfully captured from these cirrhotic samples without a loss in binding specificity, as shown in FIG. 18D. This experiment illustrates the future potential for this platform to be applied to patient samples for detection of disease-related N-glycan changes for biomarker purposes. Together, these results illustrate the development of a new MALDI MSI platform for protein-specific N-glycan analysis from biofluid samples in a clinically-relevant manner requiring minimal sample consumption.

Described here is a new mass spectrometry imaging (MSI) platform for the multiplexed detection of N-glycans in a protein-specific manner from biological samples. The development of this technique was based on a well-established protocol for enzymatic release of N-glycans from tissue sections for MALDI MSI (Powers T W et al., PloS one, 2014, 9(9), e106255; Powers T W et al., Analytical chemistry, 2013, 85(20), 9799-9806). The two-dimensional analysis with detection by MSI allows for the mapping of N-glycan signals to their carrier proteins along a slide-based antibody array. In this platform, antibodies are essential for the specific capture of glycoprotein targets from a complex biological mixture, similar to an ELISA. Yet unlike an ELISA, no secondary antibody or lectin is needed for this methodology as mass spectrometry provides sensitive and specific detection of distinct N-glycans. Antibody capture also negates the need for sample clean-up prior to MS analysis, which can be extensive (Kailemia M J et al., Analytical and bioanalytical chemistry, 2017, 409(2), 395-410; Kuzmanov U et al., BMC medicine, 2013, 11(1), 31; Song T et al., Analytical chemistry, 2015, 87(15), 7754-7762; Ruhaak L R et al., Analytical chemistry, 2008, 80(15), 6119-6126; Reiding KR et al., Analytical chemistry, 2014, 86(12), 5784-5793). Additionally, typical problems with specificity loss due to heterophilic antibodies present in diseased serum were not observed, which is an important benefit of this technique (Bolstad N et al., Best practice & research Clinical endocrinology & metabolism, 2013, 27(5), 647-661). Antibody capture has been previously used to capture a single target protein for MALDI MS analysis (Darebna P et al., Clinical chemistry, 2018, 64(9), 1319-1326; Pompach P et al., Clinical chemistry, 2016, 62(1), 270-278), however the present novel multiplexed technique can be expanded for the analysis of potentially hundreds or thousands of different N-glycoproteins in one imaging run. Each run generates an immense amount of data, as spectra showing potentially hundreds of N-glycan species are gathered localized to each glycoprotein on the array. Therefore, this method has powerful capabilities for the characterization of N-glycosylation across many target proteins simultaneously.

This new method extends the capabilities of existing N-glycan biomarker detection technologies. Lectin microarrays have been used for detection of changes in N-glycans in a biomarker setting (Chen S et al., Nature methods, 2007, 4(5), 437; Yue T et al., Molecular & Cellular Proteomics, 2009, 8(7), 1697-1707; Nagaraj V J et al., Biochemical and biophysical research communications, 2008, 375(4), 526-530; Patwa T H et al., Analytical chemistry, 2006, 78(18), 6411-6421), however the present new MSI detection method significantly increases the amount of information that can be obtained from such analyses. While lectins bind to N-glycan structural motifs, MALDI MSI detection provides N-glycans with potential compositional information. The method can be easily adapted to the use of other instrumentation, e.g., ion mobility, which will allow reporting on configuration of N-glycoforms. Additionally, MALDI MSI obtains a complete mass spectrum for each glycoprotein capture spot, allowing hundreds of N-glycan masses to be probed per glycoprotein target as opposed to a select few probed with targeted lectin analysis. Detection of the glycan heterogeneity present on each protein can be used for calculation of glycan ratios, which may represent important alterations in the overall glycosylation of a protein that can be clinically utilized (Callewaert N et al., Nature medicine, 2004, 10(4), 429; Verhelst X et al., Clinical Cancer Research, 2017, 23(11), 2750-2758). As previously mentioned, MSI analyses on tissues have been used for elucidating N-glycan changes in the presence of disease (Powers T et al., Biomolecules, 2015, 5(4), 2554-2572; Kunzke T et al., Oncotarget, 2017, 8(40), 68012; West CA et al., Journal of proteome research, 2018, 17(10), 3454-3462; Scott DA et al., PROTEOMICS-Clinical Applications, 2019, 13(1), 1800014). While tissue-based analysis is often used for prognosis and pathological examination, it is not as an accessible material for early detection of disease, as is serum or other biofluids. The present new biomarker discovery and validation platform is ready for use with readily available patient biofluids such as serum or urine.

More antibodies can be added so that more glycoproteins can be probed per analysis. This improvement will be limited by the quality of these antibodies—both in binding affinity and specificity. N-glycans present on antibodies can be removed to limit background signal. This technique is applicable to other mass spectrometry platforms for additional structural information of the detected glycans as well as more clinically-accessible MSI instruments. Mass spectrometry imaging of the peptides rather than just N-glycans can be used to confirm glycoprotein binding specificity at each antibody.

The MSI platform investigated in this study demonstrates its utility as a biomarker discovery tool as well as a new screening platform for a number of diseases in readily available clinical biofluid samples. This platform was able to detect N-glycans on glycoproteins captured from only 1 μL of human serum, illustrating its effectiveness with very minimal patient sample consumption. N-glycans and their role in disease progression are quickly becoming recognized as an important new frontier for biomedical research. However, the applications of this new technique extend beyond just N-glycans and biofluid samples: this platform could be used with liquids such as cell supernatants or probe other classes of glycans or post-translational modifications.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method for glycan analysis of at least one sample, the method comprising the steps of:
providing a substrate having a surface spotted with a plurality of different antibodies positioned at discrete locations on the substrate, wherein the plurality of different antibodies bind to a plurality of different proteins;
incubating the substrate in a blocking solution;
incubating the substrate in at least one sample, wherein the at least one sample comprises at least one protein solution derived from a biological sample, wherein the proteins in the sample bind to the plurality of different antibodies, thereby capturing the proteins;
spraying the substrate with an enzymatic releasing solution to release glycans from the captured proteins; and
scanning the substrate by mass spectrometry to detect and identify the glycans present at each discrete location, thereby identifying a plurality of glycoproteins in the sample.

2. (canceled)

3. The method of claim 1, wherein the at least one sample comprises at least one population of cultured or captured cells.

4. The method of claim 3, wherein the at least one population of cells is incubated in a fixing and rinsing agent prior to the step of spraying the substrate with an enzymatic releasing solution.

5. The method of claim 4, wherein the fixing and rinsing agent is selected from the group consisting of: formalin, Carnoy's solution, paraformaldehyde, an ethanol-based fixative, and a polyethylene glycol-based fixative.

6. The method of claim 1, wherein the substrate is a glass or plastic microscope slide or multiwell plate.

7. The method of claim 1, wherein the blocking solution is a serum.

8. The method of claim 7, wherein the serum is 1% BSA in PBS and detergent.

9. The method of claim 1, wherein the blocking solution is removed with a wash step comprising 3×PBS baths and 1×water bath.

10. The method of claim 1, wherein the at least one sample is incubated in a humidity chamber at room temperature for two hours.

11. The method of claim 1, wherein the enzymatic releasing solution comprises PNGase F.

12. The method of claim 1, wherein the mass spectrometry is selected from the group consisting of: matrix-assisted laser desorption/ionization imaging Fourier transform ion cyclotron resonance (MALDI-FTICR) mass spectrometry, matrix-assisted laser desorption/ionization time of flight (MALDI-TOF) mass spectrometry, scanning microprobe MALDI (SMALDI) mass spectrometry, infrared matrix assisted laser desorption electrospray ionization (MALD-ESI) mass spectrometry, surface-assisted laser desorption/ionization (SALDI) mass spectrometry, desorption electrospray ionization (DESI) mass spectrometry, secondary ion mass spectrometry (SIMS) mass spectrometry, and easy ambient sonic spray ionization (EASI) mass spectrometry.

13. The method of claim 12, wherein the scanning step is preceded by a step of spraying the substrate with a MALDI matrix material.

14. The method of claim 13, wherein the MALDI matrix solution is selected from the group consisting of: 2,5-dihydroxybenzoic acid, α-cyano-4-hydroxycinnamic acid, sinapinic acid, 1,5-diaminonaphthalene, and 9-aminoacridine.

15. The method of claim 1, wherein the plurality of antibodies specifically bind to a protein selected from the group consisting of: A1AT, fetuin-A, hemopexin, Apo-J, LMW Kininogen, HMW Kininogen, apo-H, transferrin, IgG, IgM, IgA, fibronectin, laminin, ceruloplasmin, fibulin, angiotensinogen, Fibrillin-1, TIMP1, thrombospondin 1, galectin-3 binding protein, complement C1 R, clusterin, galectin 1, alpha-2-macroglobulin, Vitamin D binding protein, histidine rich glycoprotein, histidine rich glycoprotein, CD109, CEA, Cathepsin, AFP, GP731, and combinations thereof.

16. The method of claim 14, wherein the antibodies are useful in detecting the presence of hepatocellular carcinoma.

17. A method for glycan analysis of at least one population of cells, the method comprising the steps of:
adhering at least one population of cells to a surface of a substrate;
fixing and rinsing the at least one population of cells;
spraying the substrate with an enzymatic releasing solution to release glycans from the captured cells; and
scanning the substrate by mass spectrometry to detect and identify the presence of glycans;
wherein the at least one population of cells comprises cultured or captured cells.

18. The method of claim 17, wherein the at least one population of cells is adhered by culturing, deposition, swabbing, smearing, or centrifugation.

19. The method of claim 17, wherein the fixing and rinsing agent is selected from the group consisting of: formalin, Carnoy's solution, paraformaldehyde, an ethanol-based fixative, and a polyethylene glycol-based fixative.

20. The method of claim 17, wherein the substrate is a glass or plastic microscope slide or multiwell plate.

21. The method of claim 17, wherein the substrate surface includes one or more of: an indium tin oxide coating, a gelatin coating, a collagen coating, a poly-l-lysine coating, a poly-ornithine coating, an extracellular matrix coating, a protein coating, and surface ionization.

22. The method of claim 17, wherein the enzymatic releasing solution comprises PNGase F.

23. The method of claim 17, wherein the mass spectrometry is selected from the group consisting of: matrix-assisted laser desorption/ionization imaging Fourier transform ion cyclotron resonance (MALDI-FTICR) mass spectrometry, matrix-assisted laser desorption/ionization time of flight (MALDI-TOF) mass spectrometry, scanning microprobe MALDI (SMALDI) mass spectrometry, infrared matrix assisted laser desorption electrospray ionization (MALD-ESI) mass spectrometry, surface-assisted laser desorption/ionization (SALDI) mass spectrometry, desorption electrospray ionization (DESI) mass spectrometry, secondary ion mass spectrometry (SIMS) mass spectrometry, and easy ambient sonic spray ionization (EASI) mass spectrometry.

24. The method of claim 23, wherein the scanning step is preceded by a step of spraying the substrate with a MALDI matrix material.

25. The method of claim 24, wherein the MALDI matrix solution is selected from the group consisting of: 2,5-dihydroxybenzoic acid, α-cyano-4-hydroxycinnamic acid, sinapinic acid, 1,5-diaminonaphthalene, and 9-aminoacridine.

26. A kit for glycan analysis of protein samples, comprising:
at least one substrate, each substrate having a surface spotted with a plurality of antibodies;
at least one blocking solution;
at least one enzymatic releasing solution; and
at least one MALDI matrix material.

27. The kit of claim 24, wherein the substrate is a glass or plastic microscope slide or multiwell plate.

28. The kit of claim 24, wherein the blocking solution is a serum.

29. The kit of claim 24, wherein the serum is 1% BSA in PBS and detergent.

30. The kit of claim 24, wherein the enzymatic releasing solution comprises PNGase F.

31. The kit of claim 24, wherein the MALDI matrix solution is α-cyano-4-hydroxycinnamic acid.
